(19) United States
(12) Patent Application Publication
(10) Pub. No.: US 2007/0293511 A1
(43) Pub. Date: Dec. 20, 2007

(54) Crf Receptor Antagonists and Methods

(75) Inventors: Zhiyong Luo, New York, NY (US); Deborah H. Slee, Cardiff, CA (US); John Edward Tellew, La Jolla, CA (US); John Williams, San Diego, CA (US); Xiaohu Zahang, San Diego, CA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SB Pharmco Puerto Rico Inc. and Neurocrine Biosciences, Inc., a Corporation

(21) Appl. No.: 10/596,646

(22) PCT Filed: Dec. 20, 2004
(86) PCT No.: PCT/IB04/04293

§ 371 Date: May 29, 2007

Related U.S. Application Data
(60) Provisional application No. 60/532,044, filed on Dec. 22, 2003.

(51) Int. Cl. A61K 31/519; A61P 1/00; A61P 25/22; A61P 25/24; A61P 9/10; C07D 487/04

(52) U.S. Cl. 514/259.3; 544/281

(57) ABSTRACT

CRF receptor antagonists are disclosed which have utility in the treatment of a variety of disorders, including the treatment of disorders manifesting hypersecretion of CRF in a warm-blooded animals, such as stroke. The CRF receptor antagonists of this invention have the following structure: 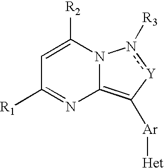
including stereoisomers, prodrugs and pharmaceutically acceptable salts thereof, wherein R₁, R₂, R₃, Y, Ar, and Het are as defined herein. Compositions containing a CRF receptor antagonist in combination with a pharmaceutically acceptable carrier are also disclosed, as well as methods for use of the same.

### CROSS-REFERENCE TO RELATED APPLICATION

This application claims priority to U.S. Provisional Application Ser. No. 60/532,044, filed Dec. 22, 2003, the entire disclosure of which is incorporated by reference herein.

### FIELD OF THE INVENTION

This invention relates generally to CRF receptor antagonists and to methods of treating disorders by administration of such antagonists to a mammal in need thereof.

### BACKGROUND OF THE INVENTION

The first corticotropin-releasing factor (CRF) was isolated from ovine hypothalami and identified as a 41-amino acid peptide (Vale et al., Science 213:1394-1397, 1981). Subsequently, sequences of human and rat CRF were isolated and determined to be identical but different from ovine CRF in 7 of the 41 amino acid residues (Rivier et al., Proc. Natl. Acad. Sci. USA 80:4851, 1983; Shibahara et al., EMBO J. 2:775, 1983).

CRF has been found to produce profound alterations in endocrine, nervous and immune system function. CRF is believed to be the major physiological regulator of the basal and stress-release of adrenocorticotropic hormone (“ACTH”), β-endorphin, and other pro-opiomelanocortin (“POMC”)-derived peptides from the anterior pituitary (Vale et al., Science 213:1394-1397, 1981). Briefly, CRF is believed to initiate its biological effects by binding to a plasma membrane receptor which has been found to be distributed throughout the brain (DeSouza et al., Science 224:1449-1451, 1984), pituitary (DeSouza et al., Methods Enzymol. 124:560, 1986; Wynn et al., Biochem. Biophys. Res. Comm. 110:602-608, 1983), adrenals (Udelsman et al., Nature 319:147-150, 1986) and spleen (Webster, E. L., and E. B. DeSouza, Endocrinology 122:609-617, 1988). The CRF receptor is coupled to a GTP-binding protein (Perrin et al., Endocrinology 118:1171-1179, 1986) which mediates CRF-stimulated increase in intracellular production of cAMP (Bilezikjian, L. M., and W. W. Vale, Endocrinology 113:657-662, 1983). The receptor for CRF has now been cloned from rat (Perrin et al., Endo 133(6):3058-3061, 1993), and human brain (Chen et al., PNAS 90(19):8967-8971, 1993; Vita et al., FEBS 335(1):1-5, 1993). This receptor is a 415 amino acid protein comprising seven membrane spanning domains. A comparison of identity between rat and human sequences shows a high degree of homology (97%) at the amino acid level.

In addition to its role in stimulating the production of ACTH and POMC, CRF is also believed to coordinate many of the endocrine, autonomic, and behavioral responses to stress, and may be involved in the pathophysiology of affective disorders. Moreover, CRF is believed to be a key intermediary in communication between the immune, central nervous, endocrine and cardiovascular systems (Crofford et al., J. Clin. Invest 90:2555-2564, 1992; Sapolsky et al., Science 238:522-524, 1987; Tilders et al., Regul. Peptides 5:77-84, 1982). Overall, CRF appears to be one of the pivotal central nervous system neurotransmitters and plays a crucial role in integrating the body's overall response to stress.

Administration of CRF directly to the brain elicits behavioral, physiological, and endocrine responses identical to those observed for an animal exposed to a stressful environment. For example, intracerebroventricular injection of CRF results in behavioral activation (Sutton et al., Nature 297:331, 1982), persistent activation of the electroencephalogram (Ehlers et al., Brain Res. 278:332, 1983), stimulation of the sympathoadrenomedullary pathway (Brown et al., Endocrinology 110:928, 1982), an increase of heart rate and blood pressure (Fisher et al., Endocrinology 110:2222, 1982), an increase in oxygen consumption (Brown et al., Life Sciences 30:207, 1982), alteration of gastrointestinal activity (Williams et al., Am. J. Physiol. 253:G582, 1987), suppression of food consumption (Levine et al., Neuropharmacology 22:337, 1983), modification of sexual behavior (Sirinathsinghji et al., Nature 305:232, 1983), and immune function compromise (Irwin et al., Am. J. Physiol. 255:R744, 1988). Furthermore, clinical data suggests that CRF may be hypersecreted in the brain in depression, anxiety-related disorders, and anorexia nervosa. (DeSouza, Ann. Reports in Med. Chem. 25:215-223, 1990). Accordingly, clinical data suggests that CRF receptor antagonists may represent novel antidepressant and/or anxiolytic drugs that may be useful in the treatment of the neuropsychiatric disorders manifesting hypersecretion of CRF.

The first CRF receptor antagonists were peptides (see, e.g., Rivier et al., U.S. Pat. No. 4,605,642; Rivier et al., Science 224:889, 1984). While these peptides established that CRF receptor antagonists can attenuate the pharmacological responses to CRF, peptide CRF receptor antagonists suffer from the usual drawbacks of peptide therapeutics including lack of stability and limited oral activity. More recently, small molecule CRF receptor antagonists have been reported Published patent documents include U.S. Pat. No. 6,313,124, WO 01/23388, and WO 97/29109, all of which disclose pyrazolopyrimidine compounds as CRF antagonists. Published application WO 98/54093 describes certain pyrazolopyrimidine compounds as tyrosine kinase inhibitors.

Due to the physiological significance of CRF, the development of biologically-active small molecules having significant CRF receptor binding activity and which are capable of antagonizing the CRF receptor remains a desirable goal. Such CRF receptor antagonists would be useful in the treatment of endocrine, psychiatric and neurological conditions or illnesses, including stress-related disorders in general.

While significant strides have been made toward achieving CRF regulation through administration of CRF receptor antagonists, there remains a need in the art for effective small molecule CRF receptor antagonists. There is also a need for pharmaceutical compositions containing such CRF receptor antagonists, as well as methods relating to the use thereof to treat, for example, stress-related disorders. The present invention fulfills these needs, and provides other related advantages.

### SUMMARY OF THE INVENTION

In brief, this invention is generally directed to CRF receptor antagonists, and more specifically to CRF receptor antagonists having the following general structure (I): 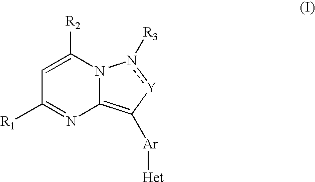
including pharmaceutically acceptable salts, esters, solvates, stereoisomers, and prodrugs thereof, wherein R₁, R₂, R₃, Y, Ar, and Het are as defined below.

The CRF receptor antagonists of this invention may have utility over a wide range of therapeutic applications, and may be used to treat a variety of disorders or illnesses, including stress-related disorders. Such methods include administering an effective amount of a CRF receptor antagonist of this invention, preferably in the form of a pharmaceutical composition, to an animal in need thereof. Accordingly, in another embodiment, pharmaceutical compositions are disclosed containing one or more CRF receptor antagonists of this invention and a pharmaceutically acceptable carrier and/or diluent.

These and other aspects of the invention will be apparent upon reference to the following detailed description. To this end, various references are set forth herein which describe in more detail certain procedures, compounds and/or compositions, and are hereby incorporated by reference in their entirety.

### DETAILED DESCRIPTION OF THE INVENTION

The present invention is directed generally to compounds useful as corticotropin-releasing factor (CRF) receptor antagonists. In a first embodiment, the CRF receptor antagonists of this invention have the following structure (I): 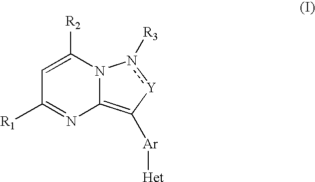
including pharmaceutically acceptable salts, esters, solvates, stereoisomers, and prodrugs thereof,

wherein:

“---” represents the second bond of an optional double bond;

R₁ is hydrogen, alkyl, substituted alkyl, —NH₂, or halogen;

R₂ is —NR₇R₈ or —OR₁₀;

R₃ is null, hydrogen, or alkyl;

Y is ═(CR₄)— or —(C═O)—;

R₄ is hydrogen, alkyl, substituted alkyl, thioalkyl, alkylsulfinyl, or alkylsulfonyl;

Ar is phenyl, phenyl optionally substituted with 1 or 2 R₅, pyridyl, or pyridyl optionally substituted with 1 or 2 R₅;

R₅ at each occurrence is alkyl, substituted alkyl, alkoxy, substituted alkoxy, cyano, halogen, alkylsulfinyl, or alkylsulfonyl;

Het is heteroaryl optionally substituted with 1 or 2 R₆;

R₆ at each occurrence is alkyl, substituted alkyl, alkoxy, substituted alkoxy, cyano, halogen, —C(O)OR₁₁, or hydroxy;

R₇ is hydrogen, alkyl, substituted alkyl, heterocycle, substituted heterocycle, heterocyclealkyl, substituted heterocyclealkyl, alkoxyalkyl, substituted alkoxyalkyl, aryl, substituted aryl, aryloxyalkyl, substituted aryloxyalkyl, arylalkyl, or substituted arylalkyl;

R₈ is alkyl, substituted alkyl, heterocycle, substituted heterocycle, heterocyclealkyl, substituted heterocyclealkyl, alkoxyalkyl, substituted alkoxyalkyl, aryl, substituted aryl, aryloxyalkyl, substituted aryloxyalkyl, arylalkyl, or substituted arylalkyl; or

R₇ and R₈, together with the nitrogen atom to which they are attached, form a heterocycle which is optionally substituted by 1, 2, or 3 R₉;

R₉ at each occurrence is hydroxy, alkylsulfonyl, alkylsulfinyl, —CH₂—OC(O)R₁₃, —C(O)OR₁₁, —C(O)NR₁₁R₁₂, alkyl, substituted alkyl, alkoxy, substituted alkoxy, arylalkyl, substituted arylalkyl, heteroarylalkyl, substituted heteroarylalkyl, aryl, substituted aryl, heterocycle, substituted heterocycle, alkoxyalkyl, or substituted alkoxyalkyl;

R₁₀ is alkyl, substituted alkyl, arylalkyl, substituted arylalkyl, heteroarylalkyl, substituted heteroarylalkyl, aryloxyalkyl, or substituted aryloxyalkyl;

R₁₁ and R₁₂ are the same or different and independently hydrogen, alkyl, substituted alkyl, heterocycle, substituted heterocycle, heterocyclealkyl, substituted heterocyclealkyl, alkoxyalkyl, substituted alkoxyalkyl, aryl, substituted aryl, aryloxyalkyl, substituted aryloxyalkyl, arylalkyl, or substituted arylalkyl; and

R₁₃ is alkyl, substituted alkyl, heterocycle, substituted heterocycle, alkoxy, substituted alkoxy.

As used herein, the above terms have the following meaning:

“Alkyl” means a straight chain or branched, noncyclic or cyclic, unsaturated or saturated aliphatic hydrocarbon containing from 1 to 10 carbon atoms, while the term “lower alkyl” has the same meaning as alkyl but contains from 1 to 6 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, —CH₂-cyclopropyl, —CH₂-cyclobutyl, —CH₂-cyclopentyl, —CH₂-cyclohexyl, and the like; while unsaturated cyclic alkyls include cyclopentenyl and cyclohexenyl, and the like. Cyclic alkyls, also referred to as “homocyclic rings,” and include di- and poly-homocyclic rings such as decalin and adamantyl. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1 butynyl, and the like.

“Alkylidenyl” represents a divalent alkyl from which two hydrogen atoms are taken from the same carbon atom, such as ═CH₂, ═CHCH₃, ═CHCH₂CH₃, ═C(CH₃)CH₂CH₃, and the like.

“Aryl” means an aromatic carbocyclic moiety such as phenyl or naphthyl.

“Arylalkyl” means an alkyl having at least one alkyl hydrogen atoms replaced with an aryl moiety, such as benzyl (i.e., —CH₂phenyl), —CH₂-(1 or 2-naphthyl), —(CH₂)₂phenyl, —(CH₂)₃phenyl, —CH(phenyl)₂, and the like.

“Aryloxyalkyl” means an aryl attached through an oxygen bridge to an alkyl (i.e., aryl-O-alkyl-) such as -methyl-O-phenyl, and such.

“Heteroaryl” means an aromatic heterocycle ring of 5- to 10-members and having at least one heteroatom selected from nitrogen, oxygen and sulfur, and containing at least 1 carbon atom, including both mono- and bicyclic ring systems. Representative heteroaryls include (but are not limited to) furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

“Heteroarylalkyl” means an alkyl having at least one alkyl hydrogen atom replaced with a heteroaryl moiety, such as —CH₂-pyridinyl, —CH₂-pyrimidinyl, and the like.

“Heterocycle” (also referred to herein as a “heterocycle ring”) means a 5- to 7-membered monocyclic, or 7- to 14-membered polycyclic, heterocycle ring which is either saturated, unsaturated or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring as well as tricyclic (and higher) heterocycle rings. The heterocycle may be attached via any heteroatom or carbon atom. Heterocycles include heteroaryls as defined above. Thus, in addition to the aromatic heteroaryls listed above, heterocycles also include (but are not limited to) morpholinyl, pyrrolidinonyl, pyrrolidinyl, piperidinyl, piperizinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocyclealkyl” means an alkyl having at least one alkyl hydrogen atom replaced with a heterocycle, such as —CH₂morpholinyl, and the like.

The term “substituted” as used herein means any of the above groups (i.e., alkyl, aryl, arylalkyl, heteroaryl, heteroarylalkyl, heterocycle or heterocyclealkyl) wherein at least one hydrogen atom is replaced with a substituent. In the case of a keto substituent (“—C(═O)—”) two hydrogen atoms are replaced. “Substituents” within the context of this invention include halogen, hydroxy, cyano, nitro, amino, alkylamino, dialkylamino, alkyl, alkoxy, thioalkyl, haloalkyl, hydroxyalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NRₐR_{b}, —NRₐC(═O)OR_{b} —NRₐSO₂R_{b}, —ORₐ, —C(═O)Rₐ, —OC(═O)ORₐ, —C(═O)ORₐ, —C(═O)NRₐR_{b}, —OC(═O)NRₐR_{b}, —SH, —SRₐ, —SORₐ, —S(═O)₂NRₐR_{b}, —S(═O)₂NRₐ, —OS(═O)₂Rₐ, —S(═O)₂ORₐ, wherein Rₐ and R_{b} are the same or different and independently hydrogen, alkyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

“Halogen” means fluoro, chloro, bromo and iodo.

“Haloalkyl” means an alkyl having at least one hydrogen atom replaced with halogen, such as trifluoromethyl and the like. Haloalkyl is a specific embodiment of substituted alkyl, wherein alkyl is substituted with one or more halogen atoms.

“Alkoxy” means an alkyl moiety attached through an oxygen bridge (i.e., —O-alkyl) such as —O-methyl, —O-ethyl, and the like.

“Thioalkyl” means an alkyl moiety attached through a sulfur bridge (i.e., —S-alkyl) such as —S-methyl, —S-ethyl, and the like.

“Alkylamino” and “dialkylamino” mean one or two alkyl moieties attached through a nitrogen bridge (i.e., —NHalkyl or —N(alkyl)(alkyl)) such as methylamino, ethylamino, dimethylamino, diethylamino, and the like.

“Hydroxyalkyl” means an alkyl substituted with at least one hydroxyl group.

“Mono- or di(cycloalkyl)methyl” represents a methyl group substituted with one or two cycloalkyl groups, such as cyclopropylmethyl, dicyclopropylmethyl, and the like.

“Alkylcarbonylalkyl” represents an alkyl substituted with a —C(═O)alkyl group.

“Alkylcarbonyloxyalkyl” represents an alkyl substituted with a —C(═O)Oalkyl group or a —OC(═O)alkyl group.

“Alkoxyalkyl” represents an alkyl substituted with a —O-alkyl group.

“Alkylthioalkyl” represents an alkyl substituted with a —S-alkyl group.

“Mono- or di(alkyl)amino represents an amino substituted with one alkyl or with two alkyls, respectively.

“Mono- or di(alkyl)aminoalkyl” represents a alkyl substituted with a mono- or di(alkyl)amino.

“Alkylsulfonyl and alkylsulfinyl” represent an alkyl substituted with a sulfonyl (—S(═O)₂—) or sulfinyl (—S(═O)—), respectively.

Embodiments of this invention presented herein are for purposes of example and not for purposes of limitation. In a first embodiment of the invention, R₃ is null and Y is ═(CR₄)— in the following structure (II), and in a further embodiment Y is —(C═O)— in the following structure (III):

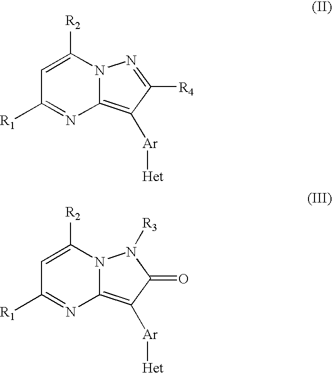

Further embodiments of this invention wherein Y is ═(CR₄)— have structure (IV) when R₂ is —NR₇R₈ and structure (V) when R₂ is —OR₁₀.

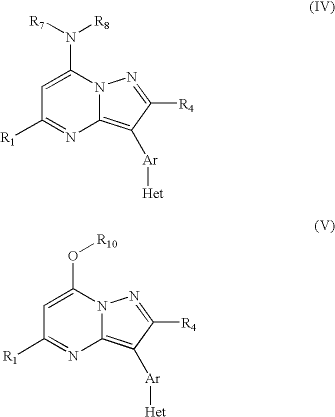

In further embodiments of this invention wherein Y is ═(CR₄)—, R₂ is —NR₇R₈ wherein R₇ and R₈, together with the nitrogen to which they are attached, form a heterocycle ring exemplified by (but not limited to) six ring atoms which can be substituted by 0, 1, 2, or 3 R₉ in the following structures (VI-VIII):

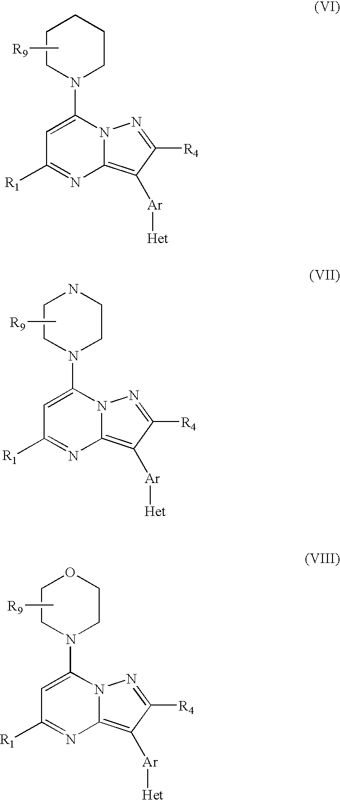

In further embodiments of this invention wherein Y is ═(CR₄)—, R₂ is —NR₇R₈ wherein R₇ and R₈, together with the nitrogen to which they are attached, form a bicyclic heterocycle ring in the following structure (IX):

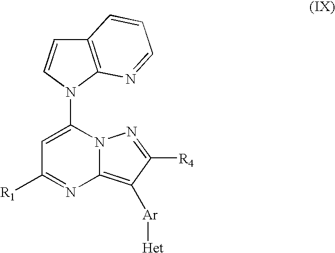

In further embodiments of this invention, Ar is phenyl substituted with 2 R₅ where each R₅ may be the same or different as shown in the following structure (X), and Het is pyridyl substituted with R₆ in the following structure (XI).

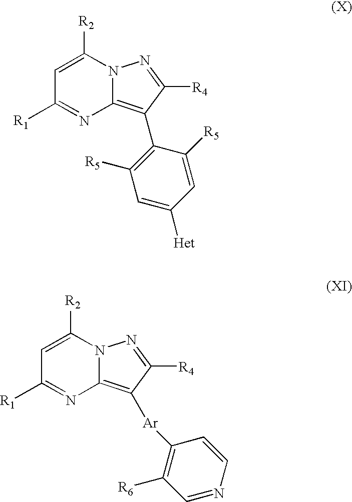

The compounds of the present invention may generally be utilized as the free base. Alternatively, the compounds of this invention may be used in the form of acid addition salts. Acid addition salts of the free base amino compounds of the present invention may be prepared by methods well known in the art, and may be formed from organic and inorganic acids. Suitable organic acids include maleic, fumaric, benzoic, ascorbic, succinic, methanesulfonic, acetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Thus, the term “pharmaceutically acceptable salt” of structure (I) is intended to encompass any and all acceptable salt forms.

In general, the compounds of structure (I) may be made according to the organic synthesis techniques known to those skilled in this field, as well as by the representative methods set forth in the Examples. For example, the synthesis of structure (I) may generally proceed according to the following Reaction Scheme 1.

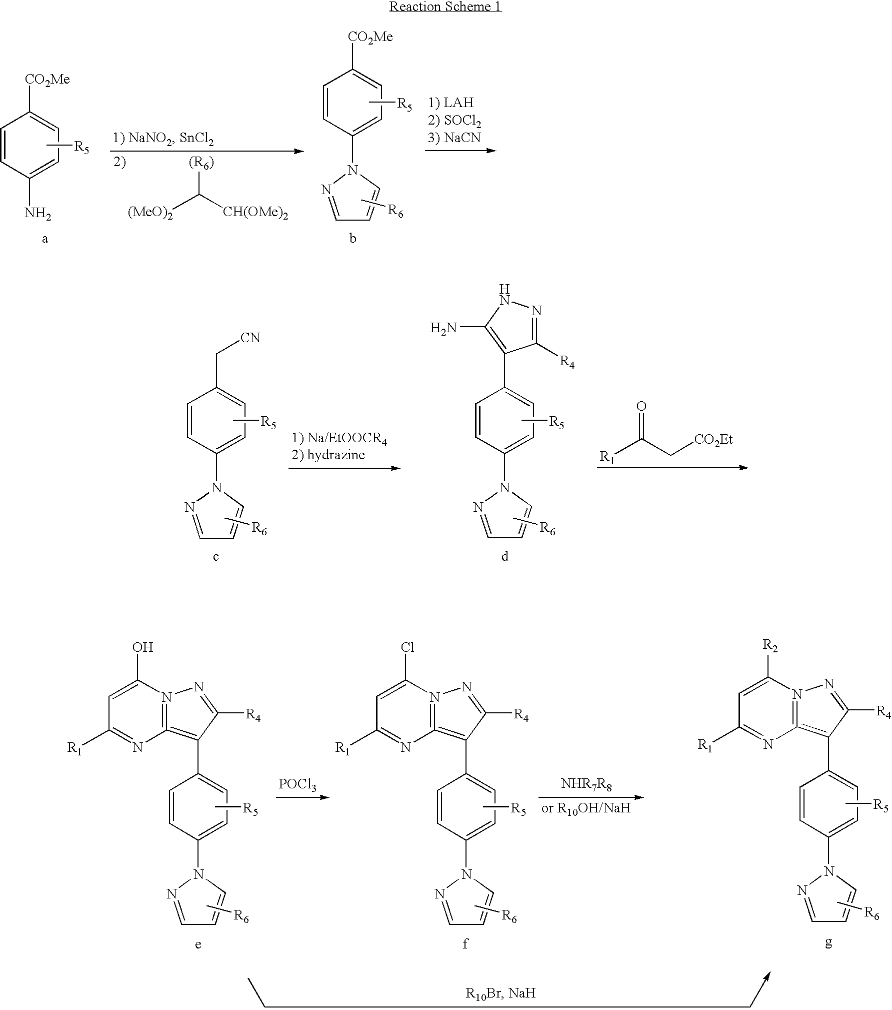

The amino functionality of 4-aminobenzoate a may be condensed with a(n) (optionally) substituted malonaldehyde to give the corresponding 4-pyrazol-1-yl benzoate b. After reaction with LAH, SOCl₂, and NaCN and conversion to the pyrazolophenylacetonitrile compound c, reaction with Na/ethyl carboxylic acid ester and hydrazine yields the bis-pyrazole d. Reaction with the appropriately substituted β-keto ester gives pyrazolopyrimidine e which reacts with POCl₃ to give the chloride f. Reaction of the chloride f with amine or alcohol gives compound g. Alternately, alkylation of e can also provide g.

The R₂ groups thus installed may be further manipulated or reacted, using standard methods known to those skilled in the art (for example oxidation/reduction, hydrolysis, and the like), to provide further examples of the invention.

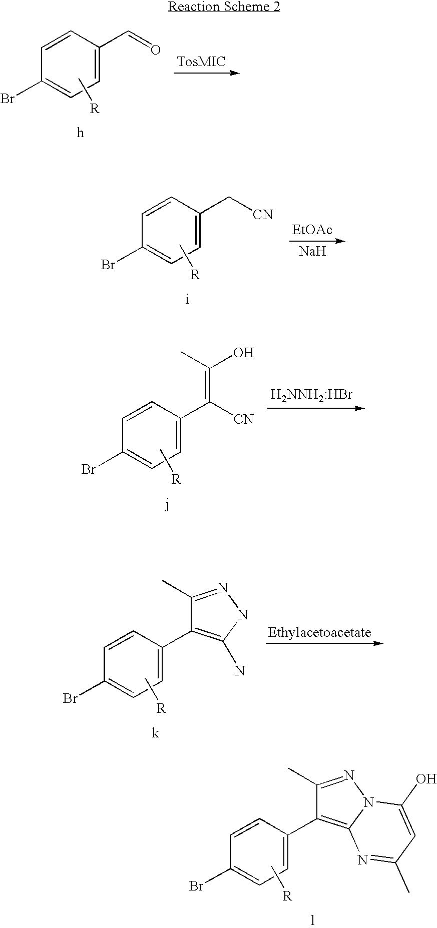

Synthetic routes available to the pyrazolopyrimidine core of the invention abound. In Reaction Scheme 2, the optionally substituted halobenzaldehyde h reacts with tosylmethyl isocyanide (TosMIC) to form the phenylacetonitrile i. Reaction of i with NaH and EtOAc gives the 3-hydroxy but-2-enenitrile j which undergoes ring closure in reaction with hydrazine HBr to give the 3-amino 2-phenyl pyrazole k. Addition of the β-keto ester gives the pyrazolo[1,5-a]pyrimidin-7-ol l. Substitution of the distal bromine with Het gives the invention.

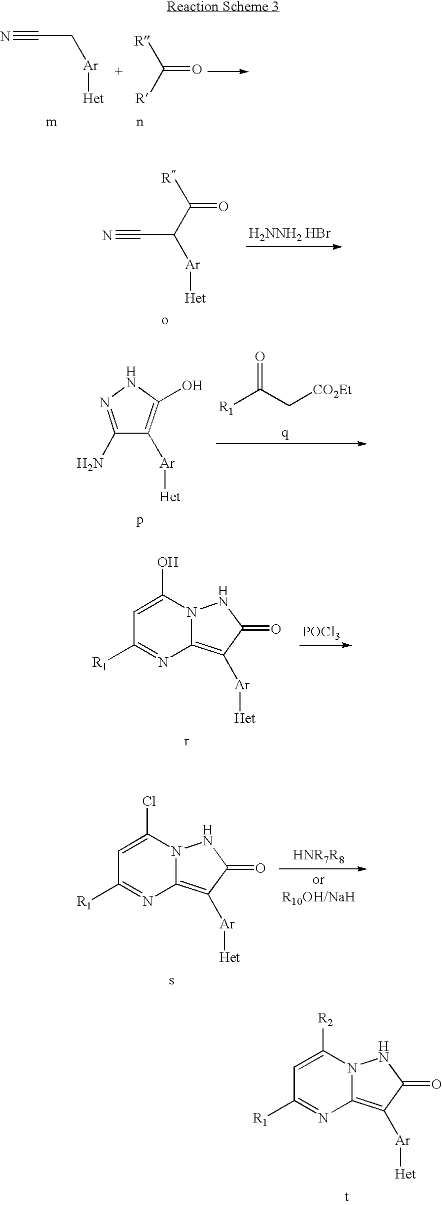

Reaction of substituted acetonitrile m with carbonyl compound n, where R′ is a good leaving group such as alkoxy, cyano, or halo, and where R″ is a group such as alkoxy, gives cyanoester o which reacts with hydrazine to give substituted pyrazole p. Reaction of p with β-keto ester q gives pyrazolopyrimidine r. Reaction with POCl₃ gives the chloride s, and reaction with amine or alcohol gives compound t.

The effectiveness of a compound as a CRF receptor antagonist may be determined by various assay methods. Suitable CRF antagonists of this invention are capable of inhibiting the specific binding of CRF to its receptor and antagonizing activities associated with CRF. A compound of structure (I) may be assessed for activity as a CRF antagonist by one or more generally accepted assays for this purpose, including (but not limited to) the assays disclosed by DeSouza et al. (J. Neuroscience 7:88, 1987) and Battaglia et al. (Synapse 1:572, 1987). As mentioned above, suitable CRF antagonists include compounds which demonstrate CRF receptor affinity. CRF receptor affinity may be determined by binding studies that measure the ability of a compound to inhibit the binding of a radiolabeled CRF (e.g., [¹²⁵I]tyrosine-CFR) to its receptor (e.g., receptors prepared from rat cerebral cortex membranes). The radioligand binding assay described by DeSouza et al. (supra, 1987) provides an assay for determining a compound's affinity for the CRF receptor. Such activity is typically calculated from the IC₅₀ as the concentration of a compound necessary to displace 50% of the radiolabeled ligand from the receptor, and is reported as a “Kᵢ” value calculated by the following equation:

$${K}_{i} = \frac{{IC}_{50}}{1 + L / {K}_{D}}$$

where L=radioligand and K_{D}=affinity of radioligand for receptor (Cheng and Prusoff, Biochem. Pharmacol. 22:3099, 1973).

In addition to inhibiting CRF receptor binding, a compound's CRF receptor antagonist activity may be established by the ability of the compound to antagonize an activity associated with CRF. For example, CRF is known to stimulate various biochemical processes, including adenylate cyclase activity. Therefore, compounds may be evaluated as CRF antagonists by their ability to antagonize CRF-stimulated adenylate cyclase activity by, for example, measuring cAMP levels. The CRF-stimulated adenylate cyclase activity assay described by Battaglia et al. (supra, 1987) provides an assay for determining a compound's ability to antagonize CRF activity. Accordingly, CRF receptor antagonist activity may be determined by assay techniques which generally include an initial binding assay (such as disclosed by DeSouza (supra, 1987)) followed by a cAMP screening protocol (such as disclosed by Battaglia (supra, 1987)).

With reference to CRF receptor binding affinities, CRF receptor antagonists of this invention may have a Kᵢ of less than 10 μM. In one embodiment of this invention, a CRF receptor antagonist has a Kᵢ of less than 1μM. In another embodiment the Kᵢ is less than 0.25 μM (i.e., 250 nM). As set forth in greater detail below, the Kᵢ values may be assayed by the methods set forth in Example 24.

The CRF receptor antagonists of the present invention may demonstrate activity at the CRF receptor site, and may be used as therapeutic agents for the treatment of a wide range of disorders or illnesses including endocrine, psychiatric, and neurological disorders or illnesses. More specifically, the CRF receptor antagonists of the present invention may be useful in treating physiological conditions or disorders arising from the hypersecretion of CRF. Because CRF is believed to be an important neurotransmitter that activates and coordinates the endocrine, behavioral and automatic responses to stress, the CRF receptor antagonists of the present invention may be used to treat neuropsychiatric disorders. Neuropsychiatric disorders which may be treatable by the CRF receptor antagonists of this invention include affective disorders such as depression; anxiety-related disorders such as generalized anxiety disorder, panic disorder, obsessive-compulsive disorder, abnormal aggression, cardiovascular abnormalities such as unstable angina and reactive hypertension; and feeding disorders such as anorexia nervosa, bulimia, and irritable bowel syndrome. CRF antagonists may also be useful in treating stress-induced immune suppression associated with various diseases states, as well as stroke. Other uses of the CRF antagonists of this invention may include treatment of inflammatory conditions (such as rheumatoid arthritis, uveitis, asthma, inflammatory bowel disease and G.I. motility), pain, Cushing's disease, infantile spasms, epilepsy and other seizures in both infants and adults, and various substance abuse and withdrawal (including alcoholism).

In another embodiment of the invention, pharmaceutical compositions containing one or more CRF receptor antagonists are disclosed. For the purposes of administration, the compounds of the present invention may be formulated as pharmaceutical compositions. Pharmaceutical compositions of the present invention comprise a CRF receptor antagonist of the present invention (i.e., a compound of structure (I)) and a pharmaceutically acceptable carrier and/or diluent. The CRF receptor antagonist is present in the composition in an amount which is effective to treat a particular disorder—that is, in an amount sufficient to achieve CRF receptor antagonist activity with acceptable toxicity to the patient. The pharmaceutical compositions of the present invention may include a CRF receptor antagonist in an amount from 0.1 mg to 250 mg per dosage depending upon the route of administration, and more typically from 1 mg to 60 mg. Appropriate concentrations and dosages can be readily determined by one skilled in the art.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets which contain, in addition to a CRF receptor antagonist, diluents, dispersing and surface active agents, binders, and lubricants. One skilled in this art may further formulate the CRF receptor antagonist in an appropriate manner, and in accordance with accepted practices, such as those disclosed in Remington's Pharmaceutical Sciences, Gennaro, Ed., Mack Publishing Co., Easton, Pa. 1990.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound.

With regard to stereoisomers, the compounds of structure (I) may have chiral centers and may occur as racemates, racemic mixtures and as individual enantiomers or diastereomers. All such isomeric forms are included within the present invention, including mixtures thereof. Furthermore, some of the crystalline forms of the compounds of structure (I) may exist as polymorphs, which are included in the present invention. In addition, some of the compounds of structure (I) may also form solvates with water or other organic solvents. Such solvates are similarly included within the scope of this invention.

In another embodiment, the present invention provides a method for treating a variety of disorders or illnesses, including endocrine, psychiatric and neurological disorders or illnesses. Such methods include administering of a compound of the present invention to a mammal in an amount sufficient to treat the disorder or illness. Such methods include systemic administration of a CRF receptor antagonist of this invention, preferably in the form of a pharmaceutical composition. As used herein, systemic administration includes oral and parenteral methods of administration. For oral administration, suitable pharmaceutical compositions of CRF receptor antagonists include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds of the present invention can be prepared in aqueous injection solutions which may contain, in addition to the CRF receptor antagonist, buffers, antioxidants, bacteriostats, and other additives commonly employed in such solutions.

In another embodiment, the present invention permits the diagnostic visualization of specific sites within the body by the use of radioactive or non-radioactive pharmaceutical agents Use of a compound of the present invention may provide a physiological, functional, or biological assessment of a patient or provide disease or pathology detection and assessment. Radioactive pharmaceuticals are employed in scintigraphy, positron emission tomography (PET), computerized tomography (CT), and single photon emission computerized tomography (SPECT.) For such applications, radioisotopes are incorporated of such elements as iodine (I) including ¹²³I (PET), ¹²⁵I (SPECT), and ¹³¹I, technetium (Tc) including ⁹⁹Tc (PET), phosphorus (P) including ³¹P and ³²P, chromium (Cr) including ⁵¹Cr, carbon (C) including ¹¹C, fluorine (F) including ¹⁸F, thallium (TI) including ²⁰¹TI, and like emitters of positron and ionizing radiation. Non-radioactive pharmaceuticals are employed in magnetic resonance imaging (MRI), fluoroscopy, and ultrasound. For such applications, isotopes are incorporated of such elements as gadolinium (Gd) including ¹⁵³Gd, iron (Fe), barium (Ba), manganese (Mn), and thallium (TI). Such entities are also useful for identifying the presence of particular target sites in a mixture and for labeling molecules in a mixture.

As mentioned above, administration of a compound of the present invention may be used to treat a wide variety of disorders or illnesses. In particular, the compounds of the present invention may be administered to a mammal for the treatment of various conditions including, for example, depression, anxiety disorder, panic disorder, obsessive-compulsive disorder, abnormal aggression, unstable angina, reactive hypertension, anorexia nervosa, bulimia, irritable bowel syndrome, stress-induced immune suppression, stroke, inflammation, pain, Cushing's disease, infantile spasms, epilepsy, and substance abuse or withdrawal.

The following examples are provided for purposes of illustration, not limitation.

### EXAMPLES

The CRF receptor antagonists of this invention may be prepared by the methods disclosed in Examples 1 to 23. Example 24 presents a method for determining the receptor binding affinity, and Example 25 discloses an assay for screening compounds of this invention for CRF-stimulated adenylate cyclase activity.

Analytical HPLC-MS Method 1

Platform: Agilent 1100 series: equipped with an auto-sampler, an UV detector (220 nM and 254 nM), a MS detector (APCI);

HPLC column: YMC ODS AQ, S-5, 5μ, 2.0×50 mm cartridge;

HPLC gradient: 1.0 mL/minute, from 10% acetonitrile in water to 90% acetonitrile in water in 2.5 minutes, maintaining 90% for 1 minute. Both acetonitrile and water have 0.025% TFA.

Analytical HPLC-MS Method 2

Platform: Agilent 1100 series: equipped with an auto-sampler, an UV detector (220 nM and 254 nM), a MS detector (APCI);

HPLC column: Phenomenex Synergi-Max RP, 2.0×50 mm column;

HPLC gradient: 1.0 mL/minute, from 5% acetonitrile in water to 95% acetonitrile in water in 13.5 minutes, maintaining 95% for 2 minute. Both acetonitrile and water have 0.025% TFA.

Analytical HPLC-MS Method 3

Platform: Agilent 1100 series: equipped with an auto-sampler, an UV detector (220 nM and 254 nM), a MS detector (electrospray);

HPLC column: XTerra MS, C₁₈, 5μ, 3.0×250 mm column;

HPLC gradient: 1.0 mL/minute, from 10% acetonitrile in water to 90% acetonitrile in water in 46 minutes, jump to 99% acetonitrile and maintain 99% acetonitrile for 8.04 minutes. Both acetonitrile and water have 0.025% TFA.

Analytical HPLC-MS Method 4

Platform: Agilent 1100 series: equipped with an auto-sampler, an UV detector (220 nM and 254 nM), a MS detector (APCI) and Berger FCM 1200 CO₂ pump module;

HPLC column: Berger Pyridine, PYR 60A, 6μ, 4.6×150 mm column;

HPLC gradient: 4.0 mL/minute, 120 bar; from 10% methanol in supercritical CO₂ to 60% methanol in supercritical CO₂ in 1.67 minutes, maintaining 60% for 1 minute. Methanol has 1.5% water. Backpressure regulated at 140 bar.

Preparative HPLC-MS

Platform: Shimadzu HPLC equipped with a Gilson 215 auto-sampler/fraction collector, UV detector and a PE Sciex API150EX mass detector;

HPLC column: BHK ODS-O/B, 5μ, 30×75 mm

HPLC gradient: 35 mL/minute, 10% acetonitrile in water to 100% acetonitrile in 7 minutes, maintaining 100% acetonitrile for 3 minutes, with 0.025% TFA.

Abbreviations:

LAH: Lithium aluminum hydride

DCM: Dichloromethane

DMSO: Dimethyl sulfoxide

EAA: Ethyl acetoacetate

LC-MS: liquid chromatography-mass spectroscopy

NaBH(OAc)₃: Sodium Triacetoxyborohydride

Pd—C: Palladium (10%) on Carbon

TFA: Trifluoroacetic acid

Tosmic: Tosylmethyl isocyanide

t_{R}: retention time (in minutes)

### Example 1

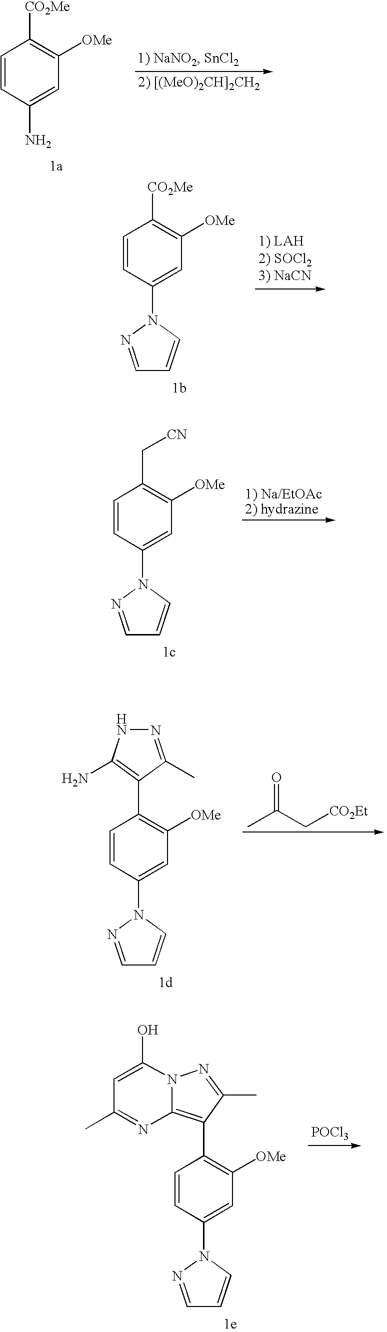 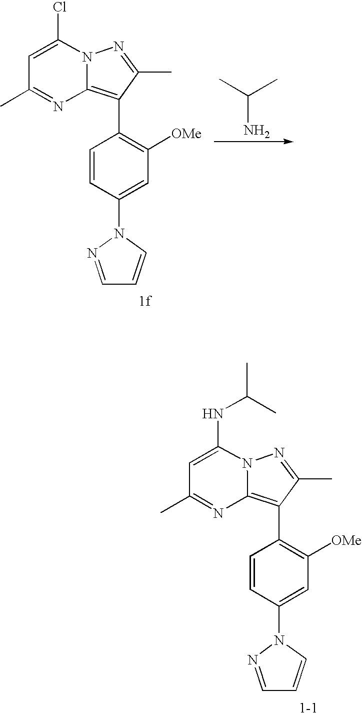 Step 1A:

To a cooled suspension of methyl 4-amino-2-methoxybenzoate 1a (6.82 g, 37.7 mmol) in 6N HCl (aqueous) was added a solution of sodium nitrite (2.60 g, 37.7 mmol) dropwise. After stirring at 0° C. for 20 min, stannous chloride dihydrate (24.7 g, 109.3 mmol) was added portionwise. The resulting suspension was stirred at 0° C. for 1.5 h prior to filtration. The collected solid was suspended in EtOH to which malonaldehyde bis(dimethyl acetal) (7.5 mL, 45.7 mmol) was added, and this reaction mixture was subjected to reflux overnight. After evaporation of EtOH, the residue was extracted between EtOAc and water, and the organic phase was dried and evaporated to dryness. The residue was passed through a silica gel plug with 25% EtOAc/hexane to give compound 1b (7.43 g) as a mixture of methyl and ethyl benzoates.

Step 1B:

To a solution of 1b (10.6 g) in dry diethyl ether (200 mL) was added LAH powder (1.74 g) slowly at 0° C. After stirring for 45 min at 0° C. the reaction mixture was decanted onto ice-water, and the aqueous phase was acidified to pH 4.0. After isolation, the alcohol was refluxed with thionyl chloride (10 mL) in DCM for 2.5 h, decanted onto ice-water, and extracted with DCM. The crude benzyl chloride was heated with NaCN (3.65 g, 74.4 mmol) in DMSO (100 mL) at 80° C. for 45 min. After removal of DMSO, compound 1c (5.98 g) obtained after chromatographic purification.

Step 1C:

To a solution of 1c (5.98 g, 28.1 mmol) in EtOAc (150 mL) was added metallic sodium (1.0 g, 43.5 mmol) portionwise, and the mixture was refluxed overnight. The resulting suspension was decanted onto ice-water and acidified to pH 4.0. The organic phase was dried, evaporated to dryness, mixed with hydrazine monohydrobromide (15.3 g, 135.4 mmol,) and refluxed for 5 h in EtOH/H₂O (6:1.) The organic phase was dried and evaporated to dryness to yield compound 1d (10.4 g.)

Step 1D:

A mixture of 1d (7.5 g, 27.9 mmol) was refluxed with ethyl acetoacetate (5.0 mL) in AcOH (100 mL) for 3 h. After evaporation of AcOH and precipitation in diethyl ether, compound 1e (10.4 g) obtained after filtration.

Step 1E:

To a suspension of 1e (2.1 g, 6.3 mmol) in acetonitrile was added POCl₃ (2.2 mL, 24.1 mmol,) and the mixture was refluxed for 5 h, decanted to ice-water, and extracted with EtOAc to yield compound 1f (1.88 g) after chromatographic purification.

Step 1F:

Displacement of the chlorine with isopropylamine followed suspension of 1f (30 mg) and excess amine in acetonitrile (0.8 mL), heating to 160° C. with microwave for 16 min, and purification with the Sciex2 preparative LC-MS system to give compound 1-1 (13.5 mg.)

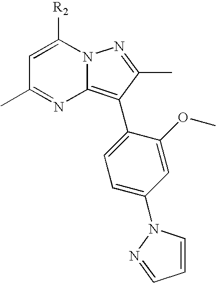

| | | | | t_{R} |
|---|---|---|---|---|
| | R₂ | MW | MS | (method) |
| 1-1 | 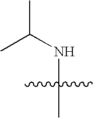 | 376.46 | 376.9 | 1.497 (4) |
| 1-2 | 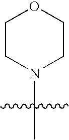 | 404.47 | 404 | 1.573 (4) |
| 1-3 | 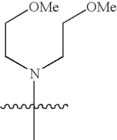 | 450.54 | 450 | 1.493 (4) |
| 1-4 | 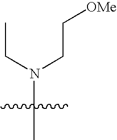 | 420.52 | 420 | 1.528 (4) |
| 1-5 | 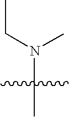 | 376.46 | 376 | 1.505 (4) |
| 1-6 | 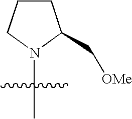 | 432.52 | 432 | 1.526 (4) |
| 1-7 | 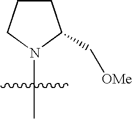 | 432.52 | 432 | 1.521 (4) |
| 1-8 | 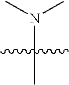 | 362.44 | 362 | 1.567 (4) |
| 1-9 | 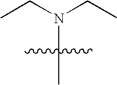 | 390.49 | 390 | 1.518 (4) |
| 1-10 | 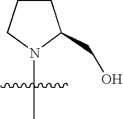 | 418.50 | 418.8 | 1.58 (4) |
| 1-11 | 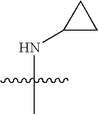 | 374.45 | 374.9 | 1.57 (4) |
| 1-12 | 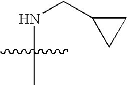 | 388.47 | 388.9 | 1.50 (4) |
| 1-13 | 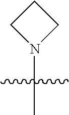 | 374.45 | 374.9 | 1.55 (4) |
| 1-14 | 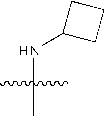 | 388.47 | 389.2 | 2.13 (1) |
| 1-15 | 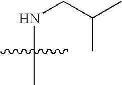 | 390.49 | 390.9 | 1.51 (4) |
| 1-16 | 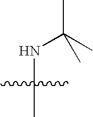 | 390.49 | 390.9 | 1.51 (4) |
| 1-17 | 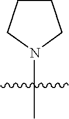 | 388.47 | 388.9 | 1.70 (4) |
| 1-18 | 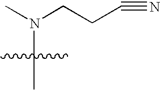 | 401.47 | 401.8 | 1.68 (4) |
| 1-19 | 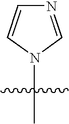 | 385.43 | 386.0 | 4.71 (2) |
| 1-20 | 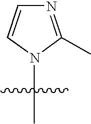 | 399.46 | 400.2 | 4.42 (2) |
| 1-21 | 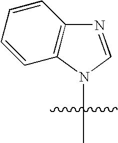 | 435.49 | 435.8 | 1.85 (4) |
| 1-22 | 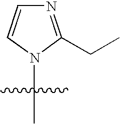 | 413.48 | 413.8 | 1.67 (4) |
| 1-23 | 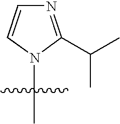 | 427.51 | 427.8 | 1.57 (4) |
| 1-24 | 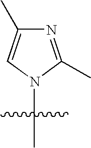 | 413.48 | 413.8 | 1.64 (4) |
| 1-25 | 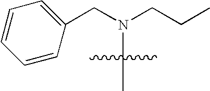 | 466.59 | 467.1 | 1.49 (4) |
| 1-26 | 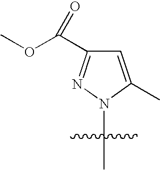 | 471.52 | 471.8 | 1.41 (4) |
| 1-27 | 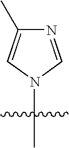 | 399.46 | 399.8 | 1.64 (4) |
| 1-28 | 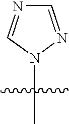 | 386.42 | 386.8 | 1.31 (4) |
| 1-29 | 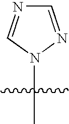 | 385.43 | 385.8 | 1.35 (4) |
| 1-30 | 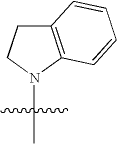 | 436.52 | 437.0 | 5.34 (2) |
| 1-31 | 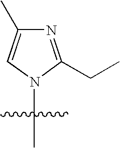 | 427.51 | 428.0 | 4.94 (2) |
| 1-32 | 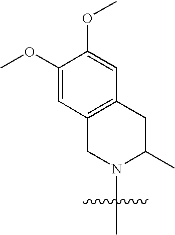 | 524.62 | 541 | 6.39 (2) |
| 1-33 | 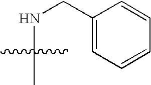 | 424.51 | 424.8 | 1.42 (4) |
| 1-34 | 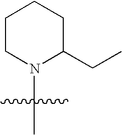 | 430.55 | 431.0 | 5.34 (2) |
| 1-35 | 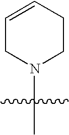 | 400.48 | 401.0 | 4.89 (2) |
| 1-36 | 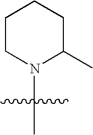 | 416.53 | 417.0 | 5.34 (2) |
| 1-37 | 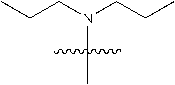 | 418.54 | 419.4 | 5.29 (2) |
| 1-38 | 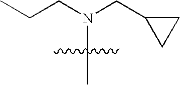 | 430.55 | 431.0 | 5.33 (2) |
| 1-39 | 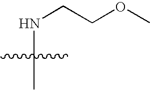 | 392.46 | 393.0 | 4.42 (2) |
| 1-40 | 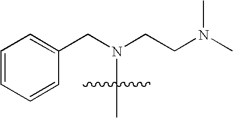 | 495.63 | 496.1 | 4.25 (2) |
| 1-41 | 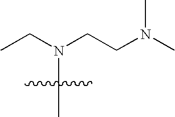 | 433.56 | 434.0 | 3.33 (2) |
| 1-42 | 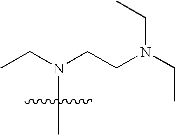 | 461.61 | 462.0 | 3.63 (2) |
| 1-43 | 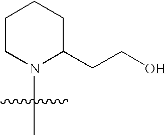 | 446.55 | 447.0 | 5.02 (2) |
| 1-44 | 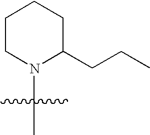 | 444.58 | 445 | 6.39 (2) |
| 1-45 | 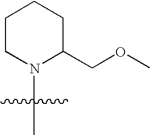 | 446.55 | 447.0 | 5.23 (2) |
| 1-46 | 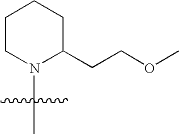 | 460.58 | 461.0 | 5.39 (2) |
| 1-47 | 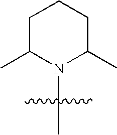 | 430.55 | 431.0 | 5.49 (2) |
| 1-48 | 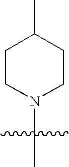 | 416.53 | 417.0 | 5.37 (2) |
| 1-49 | 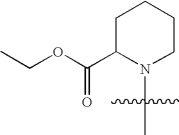 | 474.56 | 475.0 | 5.39 (2) |
| 1-50 | 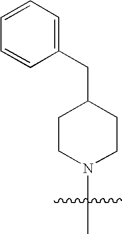 | 492.62 | 493.0 | 5.70 (2) |
| 1-51 | 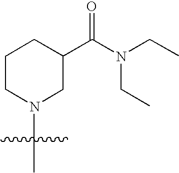 | 501.63 | 502.0 | 5.11 (2) |
| 1-52 | 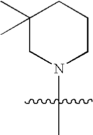 | 430.55 | 431.2 | 6.24 (2) |
| 1-53 | 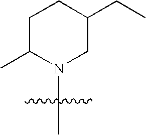 | 444.58 | 445.0 | 5.56 (2) |
| 1-54 | 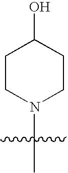 | 418.50 | 419.0 | 4.49 (2) |
| 1-55 | 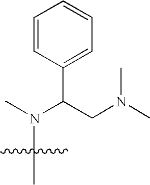 | 495.63 | 496.0 | 4.12 (2) |
| 1-56 | 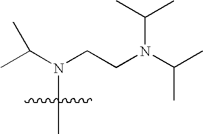 | 503.69 | 504.1 | 4.45 (2) |
| 1-57 | 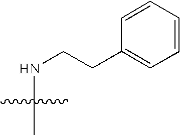 | 438.53 | 439.0 | 5.16 (2) |
| 1-58 | 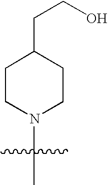 | 446.55 | 447.0 | 4.76 (2) |
| 1-59 | 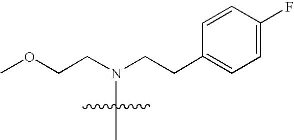 | 514.60 | 515.1 | 5.49 (2) |
| 1-60 | 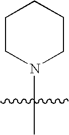 | 402.50 | 403.1 | 5.00 (2) |
| 1-61 | 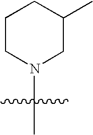 | 416.53 | 417.1 | 5.19 (2) |
| 1-62 | 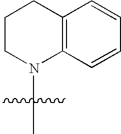 | 456.59 | 457.1 | 5.55 (2) |
| 1-63 | 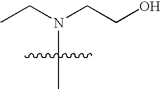 | 406.49 | 407.2 | 4.76 (2) |
| 1-64 | 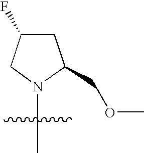 | 450.51 | 450.8 | 1.56 (4) |
| 1-65 | 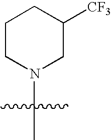 | 470.50 | 471.2 | 5.26 (2) |
| 1-66 | 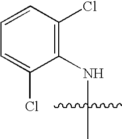 | 479.37 | 480.0 | 5.70 (2) |
| 1-67 | 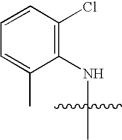 | 458.95 | 459.0 | 5.73 (2) |
| 1-68 | 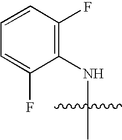 | 446.46 | 447.0 | 5.24 (2) |
| 1-69 | 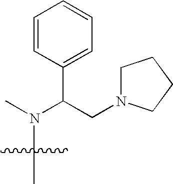 | 521.67 | 521.9 | 4.97 (2) |
| 1-70 | 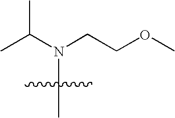 | 434.54 | 435.1 | 5.61 (2) |
| 1-71 | 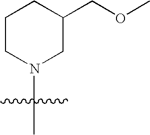 | 446.55 | 447.1 | 5.56 (2) |
| 1-72 | 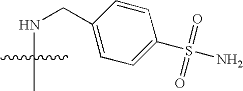 | 503.58 | 504.1 | 2.09 (4) |
| 1-73 | 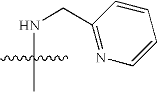 | 425.49 | 426.1 | 1.54 (4) |
| 1-74 | 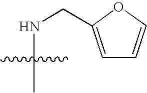 | 414.47 | 415.1 | 1.50 (4) |
| 1-75 | 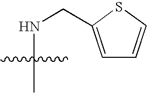 | 430.53 | 431.1 | 1.56 (4) |
| 1-76 | 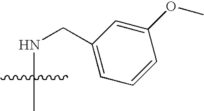 | 454.53 | 455.1 | 1.54 (4) |
| 1-77 | 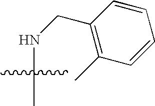 | 438.53 | 439.1 | 1.53 (4) |
| 1-78 | 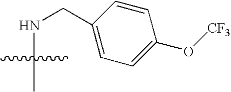 | 508.50 | 509.1 | 1.49 (4) |
| 1-79 | 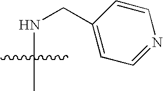 | 425.49 | 426.1 | 1.85 (4) |
| 1-80 | 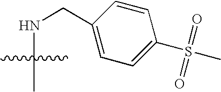 | 502.60 | 503.1 | 1.80 (4) |
| 1-81 | 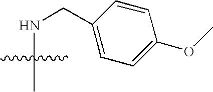 | 454.53 | 455.0 | 5.53 (2) |
| 1-82 | 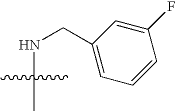 | 442.50 | 443.1 | 1.71 (4) |
| 1-83 | 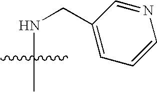 | 425.49 | 426 | 3.41 (2) |
| 1-84 | 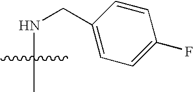 | 442.50 | 443.1 | 1.52 (4) |
| 1-85 | 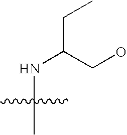 | 404.51 | 405.1 | 1.88 (4) |
| 1-86 | 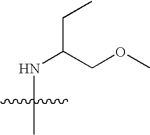 | 420.51 | 421.2 | 1.87 (4) |
| 1-87 | 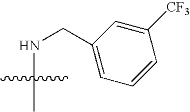 | 492.50 | 493.1 | 1.50 (4) |
| 1-88 | 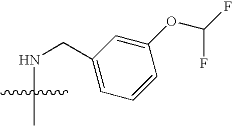 | 490.51 | 491.1 | 1.54 (4) |
| 1-89 | 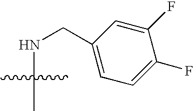 | 460.49 | 461.1 | 1.56 (4) |
| 1-90 | 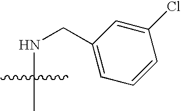 | 458.95 | 459.1 | 1.53 (4) |
| 1-91 | 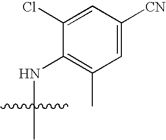 | 483.96 | 484.1 | 1.58 (4) |
| 1-92 | 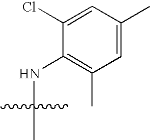 | 472.98 | 473.1 | 1.49 (4) |
| 1-93 | 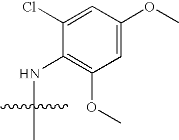 | 504.98 | 505.1 | 1.50 (4) |
| 1-94 | 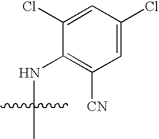 | 504.38 | 505.0 | 1.53 (4) |
| 1-95 | 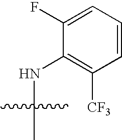 | 496.47 | 497.1 | 1.46 (4) |
| 1-96 | 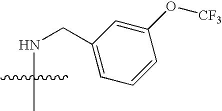 | 508.50 | 509.0 | 6.44 (2) |
| 1-97 | 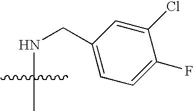 | 476.94 | 477.0 | 1.55 (4) |
| 1-98 | 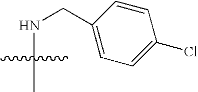 | 458.95 | 459.1 | 1.55 (4) |
| 1-99 | 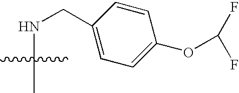 | 490.51 | 491.0 | 5.95 (2) |
| 1-100 | 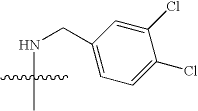 | 493.40 | 493.1 | 1.58 (4) |
| 1-101 | 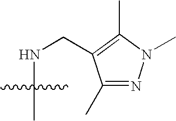 | 456.55 | 457.2 | 1.47 (4) |
| 1-102 | 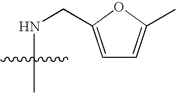 | 428.49 | 429.1 | 1.43 (4) |
| 1-103 | 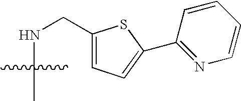 | 507.62 | 508.1 | 1.59 (4) |
| 1-104 | 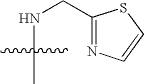 | 431.52 | 432.1 | 1.53 (4) |
| 1-105 | 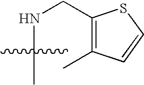 | 444.56 | 445.1 | 1.48 (4) |
| 1-106 | 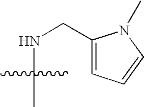 | 427.51 | 428.1 | 1.50 (4) |
| 1-107 | 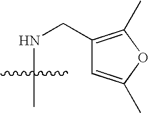 | 442.52 | 443.1 | 1.45 (4) |
| 1-108 | 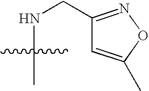 | 429.48 | 430.1 | 1.50 (4) |
| 1-109 | 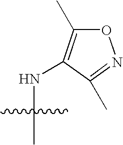 | 429.48 | 430.0 | 4.37 (2) |
| 1-110 | 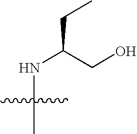 | 406.49 | 407.0 | 4.44 (2) |

### Example 2

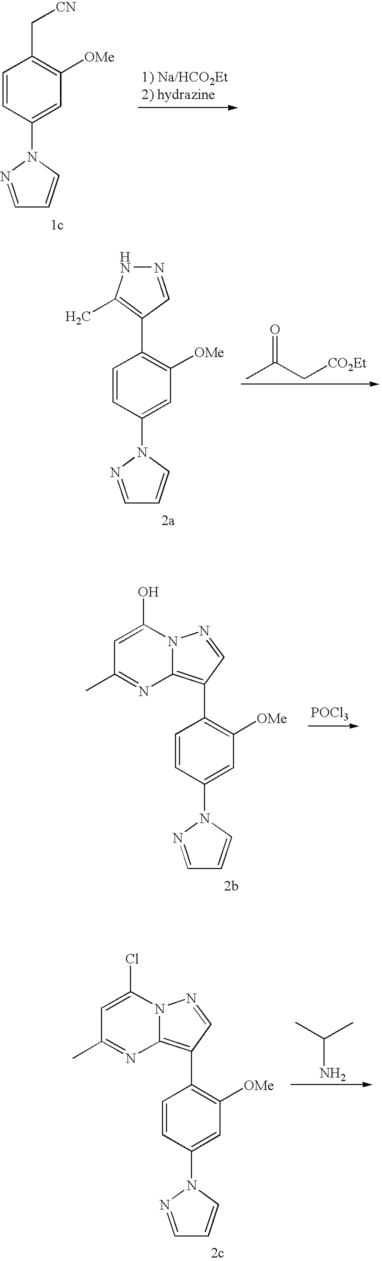 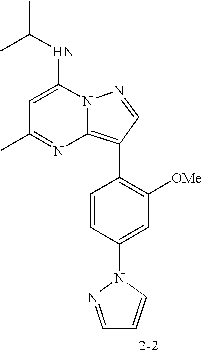 Step 2A:

In order to introduce hydrogen at position R₄ of the invention, the synthetic scheme of Example 1 was modified at Step 1C to give the synthetic scheme of Example 2. To a solution of 1c (1.0 g) in HCO₂Et (20 mL) was added metallic sodium (0.13 g) portionwise, and the mixture was refluxed for 1.5 h. The resulting suspension was decanted onto ice-water and acidified to pH 4.0. The organic phase was dried, evaporated to dryness, mixed with hydrazine monohydrobromide (1.58 g) and refluxed for 1 h in EtOH/H₂O (6:1.) After evaporation of EtOH, the mixture was extracted between EtOAc and NaOH (aq.) The organic phase was dried and evaporated to dryness to yield compound 2a (1.20 g.)

Step 2B:

A mixture of 2a (1.2 g) was refluxed with ethyl acetoacetate (1.0 mL) in AcOH (30 mL) for 2 h. After evaporation of AcOH and precipitation in diethyl ether, compound 2b (1.0 g) obtained after filtration.

Step 2C:

To a suspension of 2b (1.0 g) in acetonitrile (30 mL) was added POCl₃ (2.0 mL,) and the mixture was refluxed overnight, decanted to ice-water, and extracted with EtOAc to yield compound 2c (0.92 g) after chromatographic purification.

Step 2D:

Displacement of the chlorine with isopropylamine followed suspension of 2c (30 mg) and excess amine in acetonitrile (0.8 mL), heating to 160° C. with microwave for 16 min, and purification with the Sciex2 preparative LC-MS system to yield compound 2-2 (14.8 mg.) Depending on the reacting amine, reaction of 2c with amine gave the compounds listed in the following table.

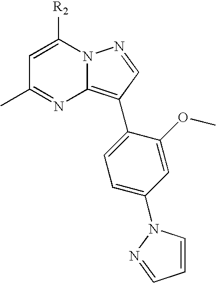

| | | | | t_{R} |
|---|---|---|---|---|
| | R₂ | MW | MS | (method 4) |
| 2-1 | 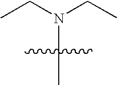 | 376.46 | 377 | 1.577 |
| 2-2 | 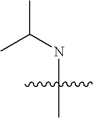 | 362.44 | 363 | 1.512 |
| 2-3 | 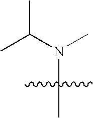 | 362.44 | 363 | 1.650 |
| 2-4 | 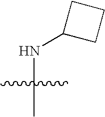 | 374.45 | 375 | 1.611 |
| 2-5 | 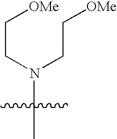 | 436.51 | 437 | 1.451 |
| 2-6 | 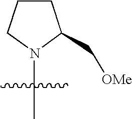 | 418.50 | 419 | 1.564 |

### Example 3

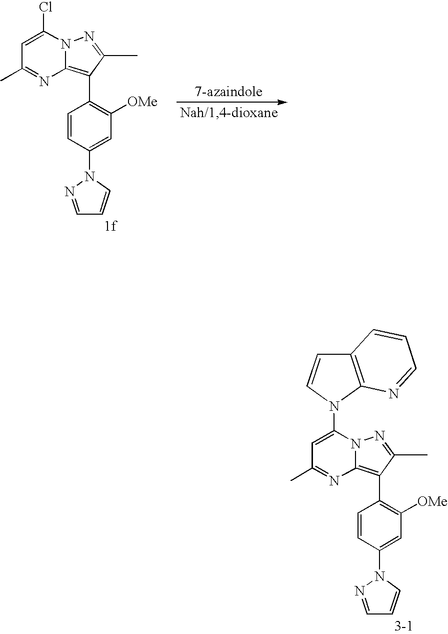 Step 3A:

To a solution of 7-azainole (24 mg) in dry 1,4-dioxane was added NaH (12 mg) with stirring for 15 min. Compound 1f (35 mg) was added with stirring overnight. Preparative LC-MS purification gave compound 3-1 (6.1 mg.) Depending on the reacting amine, reaction of 1f with amine gave the compound(s) listed in the following table.

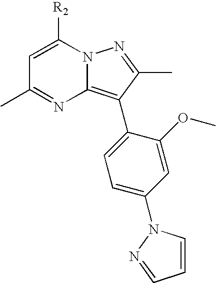

| | | | | t_{R} |
|---|---|---|---|---|
| | R₂ | MW | MS | (method 4) |
| 3-1 | 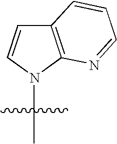 | 435.49 | 435.8 | 1.601 (4) |
| 3-2 | 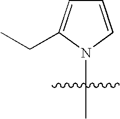 | 412.50 | 413.1 | 2.57 (1) |

### Example 4

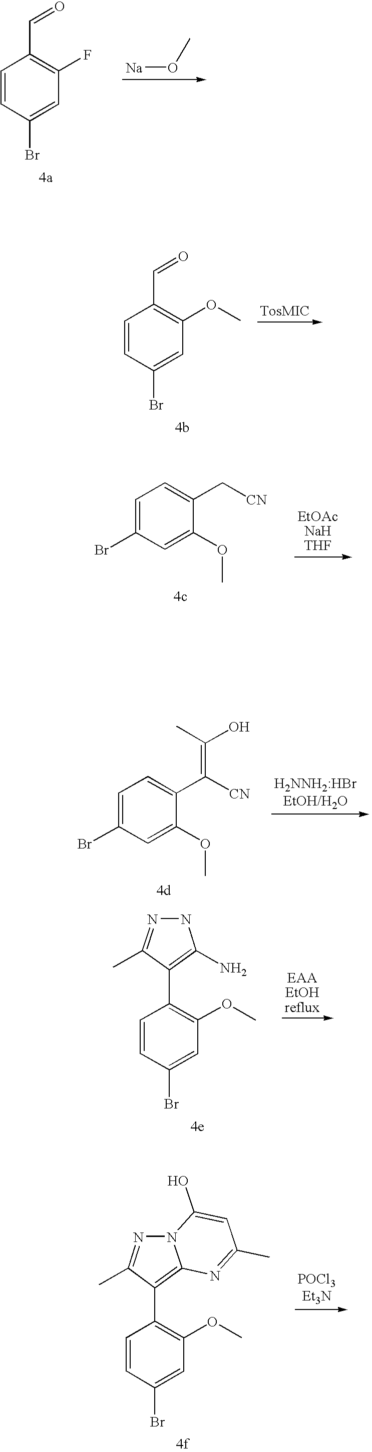 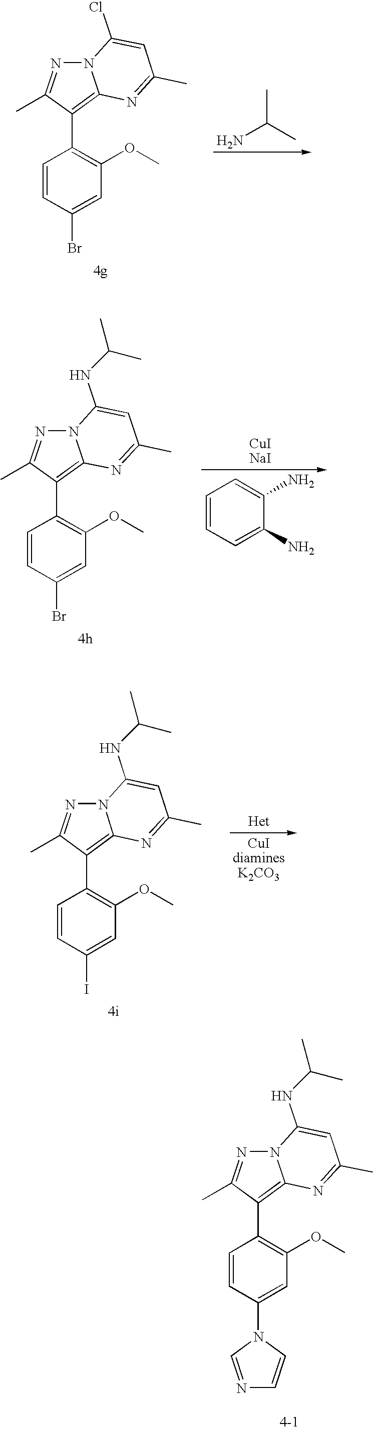 Step 4A:

Compound 4a (40 g, Aldrich,) was dissolved in 200 mL THF. Sodium methoxide solution (48 mL, 25% in MeOH) was added dropwise, and the reaction mixture was stirred at room temperature for 6 hr. Following quenching with 150 mL water, the mixture was neutralized with 4N HCl and extracted with DCM. The organic layer was dried under sodium sulfate, concentrated, and purified by silica gel chromatography to give compound 4b (17.7 g.)

Step 4B:

A suspension of potassium t-butyloxide (7.3 g) in DME (40 mL) was chilled to −50° C. under nitrogen. Tosmic (9.1 g) in DME (40 mL) was added dropwise with maintenance of temperature. To the reaction mixture was introduced compound 4b (10 g) with stirring for 30 min. MeOH (100 mL) was added, and the reaction mixture was refluxed for 30 min. After removal of most of the DME and MeOH, the residue was resuspended in water (100 mL) and ethyl acetate (100 mL) and neutralized with acetic acid. The organic layer was washed with brine, dried under sodium sulfate, concentrated, and purified with silica gel chromatography to give compound 4c (7.0 g.)

Step 4C:

Under nitrogen, to compound 4c (6.25 g) dissolved in THF (80 mL) was added NaH (2.3 g, 60% in oil) and ethyl acetate (1.5 mL.) The mixture was gently heated with a handheld heat gun until small bubbles evolved from the mixture. Ethyl acetate was added to keep the reflux. The reaction was kept at room temperature for one hour, quenched with water (100 mL,) and extracted with diethyl ether (100 mL.) The aqueous solution was neutralized with 4N HCl and extracted twice with ethyl acetate (100 mL aliquots.) The organic layer was dried over sodium sulfate and concentrated to give compound 4d (6.5 g.)

Step 4D:

Compound 4d (12.1 g) and hydrazine:HBr (5.61 g) were dissolved in EtOH:H₂O (100 mL, 9:1 mixture,) and the mixture was refluxed for 2 hr. After concentration, the mixture was partitioned between ethyl acetate (200 mL) and saturated sodium bicarbonate (150 mL.) The organic layer was dried under sodium sulfate and concentrated to give compound 4e (12.2 g.)

Step 4E:

Compound 4e (12.2 g) and acetyl acetate (9.06 g) were mixed with ethanol (50 mL) and the mixture was refluxed overnight. Upon cooling, crystals formed and were harvested. The filtrate was further treated with diethyl ether to afford compound 4f (10.76 g.)

Step 4F:

Compound 4f (2.0 g) was dissolved in POCl₃ (1.34 mL, 14.44 mmol) and Et₃N (1.6 mL) to which dioxane (10 mL) was added, and the mixture was refluxed 2 hr. The reaction mixture was poured over ice and sodium carbonate was added to adjust to pH 7. Extraction with EtOAc, drying over MgSO4, filtration and evaporation were followed by chromatography to give compound 4g (2.0 g.)

Step 4G:

To compound 4g (1.0 g) in ethanol (10 mL) was added isopropylamine (2.0 eq.) The reaction mixture was heated overnight in a pressure vessel. Removal of ethanol and column chromatography yielded compound 4h (0.95 g.) Using N-ethyl-N-methoxyethylamine in place of isopropylamine gave compound 4h.1. Using (S)-2-(methoxymethyl)pyrrolidine in place of isopropylamine gave compound 4.h.2.

Step 4H:

To compound 4h (0.8 g) in dioxane (20 mL) was added CuI (0.03 g,) NaI (0.63 g,) and trans-1,2-diaminocyclohexane (0.0036 mL), and this mixture was heated overnight at 110° C. The reaction mixture was filtered, the dioxane was removed, and the residue was dissolved in EtOAc and washed with brine. Filtration through silica gel yielded compound 4i (0.81 g.)

Step 4I:

To compound 4i (40 mg) in dioxane (2 mL) was added imidazole (1.5 eq), CuI (26.8 mg,) K₂CO₃ (53.2 mg,) trans-1,2-diaminocyclohexane (0.0015 mL,) and N,N-dimethylenediamine (0.0014 mL,) and this reaction mixture was heated to 110° C. overnight. The reaction mixture was filtered and purified via preparative HPLC to give compound 4-1 (8.3 mg.) Depending on the reagents employed in this synthetic scheme for the R₂ and Het positions of the invention, the compounds in the following table were obtained.

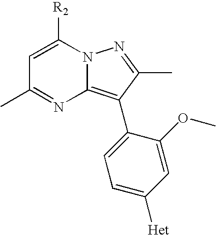

| | | | | | t_{R} |
|---|---|---|---|---|---|
| | R₂ | Het | MW | MS | (method) |
| 4-1 | 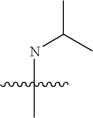 | 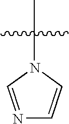 | 376.46 | 377 | 1.671 (4) |
| 4-2 | 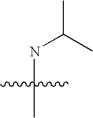 | 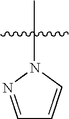 | 390.49 | 391 | 1.554 (4) |
| 4-3 | 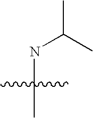 | 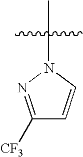 | 444.46 | 445 | 2.318 (4) |
| 4-4 | 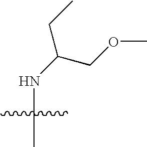 | 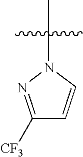 | 488.51 | 489 | 1.478 (4) |
| 4-5 | 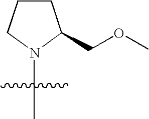 | 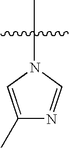 | 446.55 | 447 | 3.830 (2) |
| 4-6 | 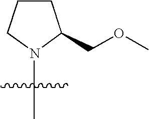 | 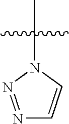 | 433.51 | 434 | 5.640 (2) |
| 4-7 | 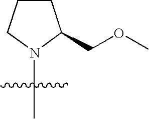 | 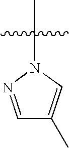 | 446.55 | 447 | 5.900 (2) |
| 4-8 | 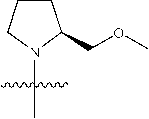 | 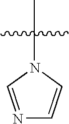 | 432.52 | 433 | 3.730 (2) |
| 4-9 | 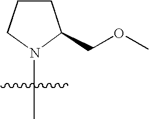 | 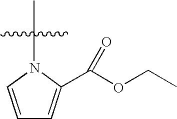 | 503.60 | 504 | 5.630 (2) |
| 4-10 | 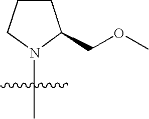 | 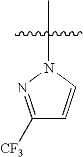 | 500.52 | 501 | 5.650 (2) |
| 4-11 | 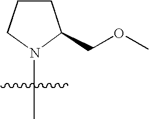 | 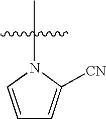 | 456.55 | 457 | 5.260 (2) |
| 4-12 | 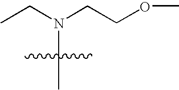 | 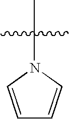 | 419.53 | 420 | 6.21 (2) |
| 4-13 | 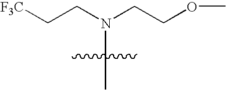 | 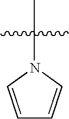 | 487.52 | 488.1 | 27.97 (3) |

### Example 5

### Preparation of Intermediate

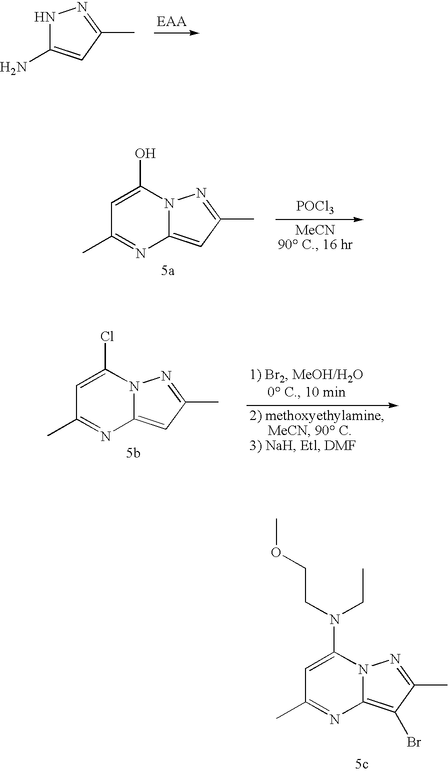 Step 5A:

A solution of 3-amino-5-methylpyrazole (20.0 g, 206 mmol), ethyl acetoacetate (32.0 g, 247 mmol), acetic acid (6 mL), and dioxane (150 mL) was refluxed for 16 h. A white solid precipitated, which was collected by filtration. The filter cake was washed with ether to provide 5a (29.0 g, 86%) as a white solid.

Step 5B:

To a suspension of 5a (9.0 g, 55 mmol) in acetonitrile (50 mL) was added phosphorous oxychloride (12.7 g, 83 mmol). The mixture was stirred and heated in a sealed tube at 90° C. for 16 h. The cooled reaction mixture was poured onto ice. The mixture was neutralized with solid sodium bicarbonate, then was extracted with ethyl acetate. The combined organic layers were dried over sodium sulfate, filtered, and concentrated to a dark brown oil. The crude product was purified by silica gel chromatography using 30% ethyl acetate in hexanes as eluant, providing 5b (9.9 g, 99%) as a white solid.

Step 5C:

Bromine (5.3 g, 33 mmol) was added dropwise to a solution of 5b (6.7 g, 37 mmol) in 1:1 methanol/water (60 ml) at 0° C. After 10 min, the mixture was filtered to collect the precipitate that had formed. The solid was washed with cold methanol, then one half of the resulting orange solid was suspended in 50 mL acetonitrile. 2-Methoxyethylamine (2.5 g, 33 mmol) was added and the mixture was stirred and heated in a sealed tube at 90° C. for 16 h. The mixture was concentrated, then the residue was taken up in dry DMF (25 ml) and treated with sodium hydride (2.1 g of 60% dispersion in mineral oil, 53 mmol) and iodoethane (8.1 g, 52 mmol). The mixture was heated at 85° C. for 16 h, then was heated at reflux for 16 h. 100 ml water was added, then the mixture was extracted with ethyl acetate. The combined organic extracts were dried over sodium sulfate, filtered, and concentrated, and the residue was purified by silica gel chromatography, eluting with 3:1 hexanes/ethyl acetate to provide 5c (0.95 g, 16% yield).

Similarly prepared were:

5d by substituting diethylamine in place of 2-methoxyethylamine and omitting the alkylation step;

5e by substituting di-N-propylamine in place of 2-methoxyethylamine and omitting the alkylation step;

5f by substituting N-propylbenzylamine in place of 2-methoxyethylamine and omitting the alkylation step;

5g by substituting N′-benzyl-N,N-dimethylethylenediamine in place of 2-methoxyethylamine and omitting the alkylation step.

### Example 6

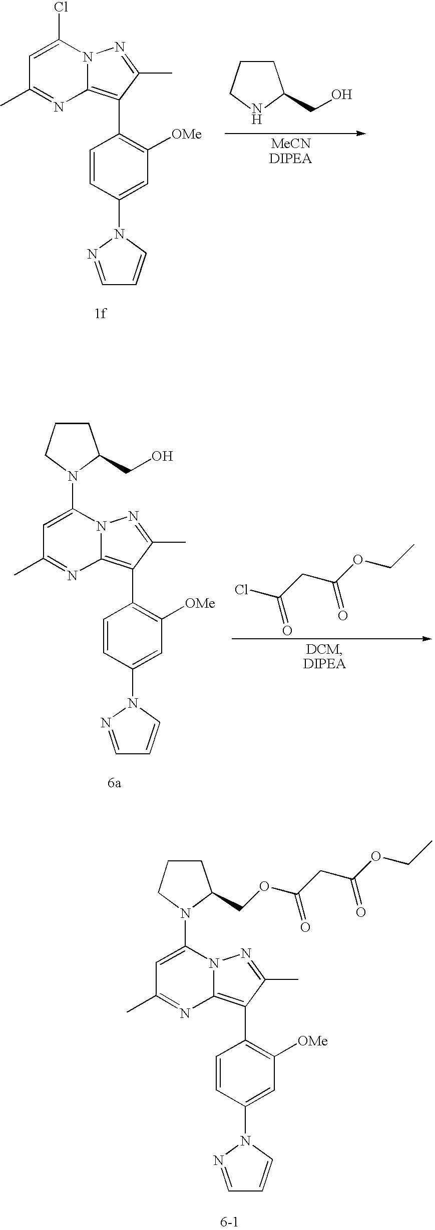 Step 6A:

A suspension of compound 1f (706 mg, 2.0 mmol), (S)-prolinol (263 mg, 2.6 mmol), and DIPEA (390 mg, 3.0 mmol) in acetonitrile (20 mL) was heated at reflux for 3 h. The solvent was evaporated, water was added, and the mixture was extracted with chloroform. The combined organic extracts were dried over sodium sulfate, filtered, and concentrated to provide 6a (720 mg).

Step 6B:

Ethyl malonyl chloride (10 mg, 0.06 mmol) was added to a solution of 6a (20 mg, 0.05 mmol), DIPEA (10 mg, 0.08 mmol), and DMAP (1 mg) in chloroform (0.5 mL) at rt. The mixture was allowed to sit for 16 h, then the solvent was evaporated. The residue was taken up in methanol and purified directly by preparative HPLC/MS, providing 6-1 (21 mg) as a TFA salt.

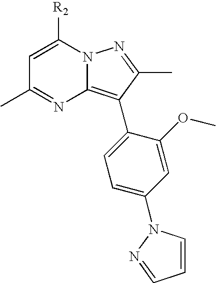

| | | | | t_{R} |
|---|---|---|---|---|
| | R₂ | MW | MS | (method 4) |
| 6-1 | 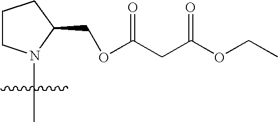 | 532.60 | 532.7 | 1.53 |
| 6-2 | 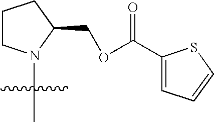 | 528.63 | 528.7 | 1.54 |
| 6-3 | 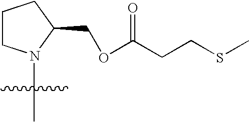 | 520.65 | 520.8 | 1.51 |
| 6-4 | 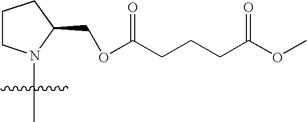 | 546.63 | 546.8 | 1.51 |
| 6-5 | 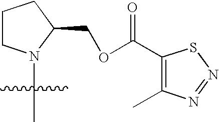 | 544.64 | 544.7 | 1.54 |
| 6-6 | 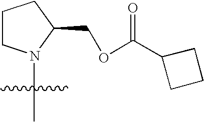 | 500.60 | 500.8 | 1.54 |
| 6-7 | 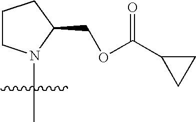 | 486.57 | 486.8 | 1.43 |
| 6-8 | 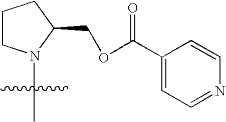 | 523.59 | 523.7 | 1.50 |
| 6-9 | 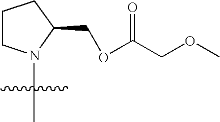 | 490.56 | 490.8 | 1.48 |
| 6-10 | 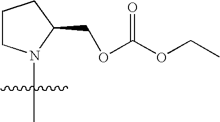 | 490.56 | 490.8 | 1.46 |
| 6-11 | 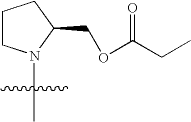 | 474.56 | 474.8 | 1.55 |
| 6-12 | 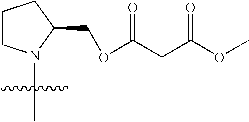 | 518.57 | 518.8 | 1.64 |
| 6-13 | 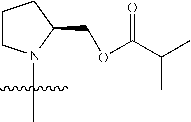 | 488.59 | 488.8 | 1.51 |
| 6-14 | 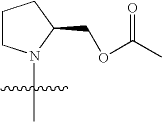 | 460.54 | 460.8 | 1.52 |

### Example 7

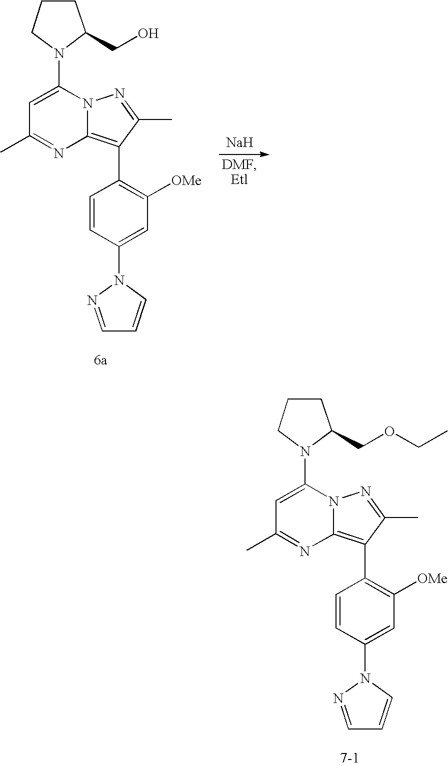 Step 7A:

Sodium hydride (4 mg of 60% dispersion in mineral oil, 0.10 mmol, 2 eq) was added to a solution of 6a (20 mg, 0.05 mmol) in DMF (0.5 mL) and the mixture was stirred at rt for 15 min. Iodoethane (16 mg, 0.10 mmol, 2 eq) was added and the mixture was heated at 75° C. in a sealed vial for 3 h. The mixture was diluted with methanol and purified directly by preparative HPLC/MS, providing 7-1 (13 mg) as a TFA salt.

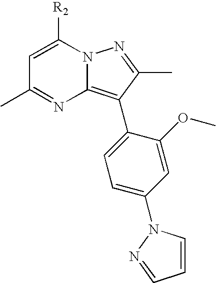

| | | | | t_{R} |
|---|---|---|---|---|
| | R₂ | MW | MS | (method 4) |
| 7-1 | 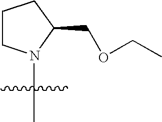 | 446.55 | 446.8 | 1.60 |
| 7-2 | 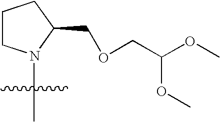 | 506.60 | 506.8 | 1.48 |
| 7-3 | 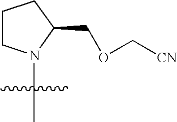 | 456.55 | 456.8 | 1.51 |
| 7-4 | 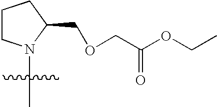 | 504.59 | 504.8 | 1.46 |

### Example 8

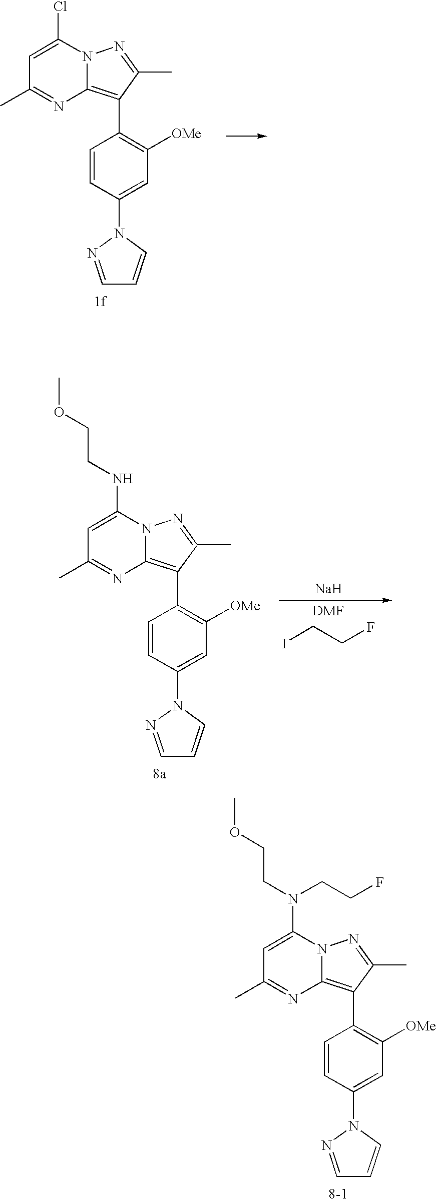 Step 8A:

A solution of 1f (50 mg, 0.14 mmol) and 2-methoxyethylamine (0.040 mL, 0.46 mmol) in acetonitrile (2 mL) was heated in a sealed tube in a microwave reactor at 150° C. for 1000 seconds. Ethyl acetate was added and the mixture was washed with water and brine. The organic layer was dried over sodium sulfate, filtered, and concentrated to provide 8a (45 mg) as an oil.

Step 8B:

Sodium hydride (15 mg of 60% dispersion in mineral oil, 0.38 mmol) was added to a solution of 8a (45 mg, 0.11 mmol) in DMF (0.5 mL) and the mixture was stirred at rt for 15 min. 1-Fluoro-2-iodoethane (30 mg, 0.17 mmol) was added and the mixture was heated at 80° C. in a sealed vial for 3 h. Ethyl acetate was added and the mixture was washed with water and brine. The organic layer was dried over sodium sulfate, filtered, and concentrated. The residue was purified by silica gel chromatography, eluting with 1:1 hexanes/ethyl acetate to provide 8-1 (6 mg).

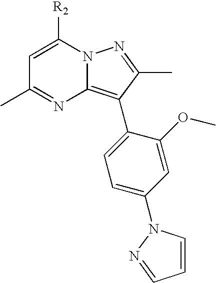

| | R₂ | MW | MS | t_{R} | |
|---|---|---|---|---|---|
| 8-1 | 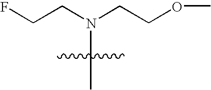 | 438.50 | 438.8 | 1.44 | 4 |
| 8-2 | 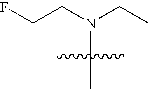 | 408.48 | 408.8 | 1.51 | 4 |
| 8-3 | 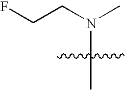 | 394.45 | 394.8 | 1.60 | 4 |
| 8-4 | 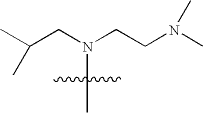 | 461.61 | 462.1 | 4.18 | 2 |
| 8-5 | 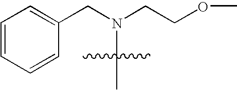 | 482.58 | 483.4 | 6.42 | 2 |
| 8-6 | 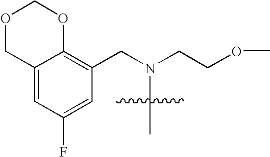 | 558.61 | 559.0 | 6.46 | 2 |
| 8-7 | 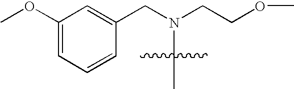 | 512.61 | 513.0 | 6.46 | 2 |
| 8-8 | 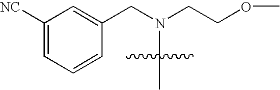 | 507.60 | 508.0 | 6.09 | 2 |
| 8-9 | 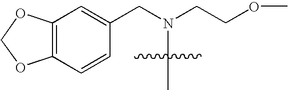 | 526.59 | 527.0 | 6.38 | 2 |
| 8-10 | 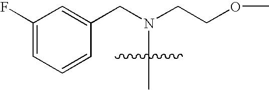 | 500.57 | 501.4 | 5.88 | 2 |
| 8-11 | 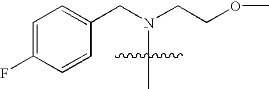 | 500.57 | 501.3 | 6.59 | 2 |
| 8-12 | 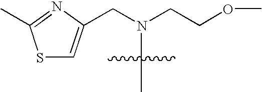 | 503.63 | 504.0 | 5.71 | 2 |
| 8-13 | 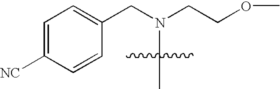 | 507.60 | 508.0 | 5.64 | 2 |
| 8-14 | 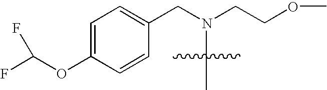 | 548.59 | 549.0 | 5.76 | 2 |
| 8-15 | 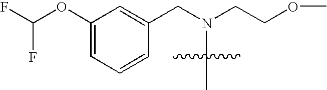 | 548.59 | 549.0 | 5.54 | 2 |
| 8-16 | 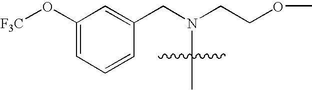 | 566.58 | 566.9 | 5.75 | 2 |
| 8-17 | 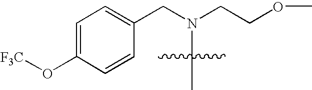 | 566.58 | 567.0 | 5.62 | 2 |
| 8-18 | 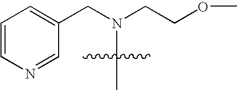 | 483.57 | 484.0 | 4.17 | 2 |
| 8-19 | 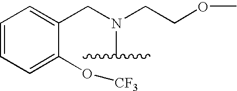 | 566.58 | 567.0 | 5.88 | 2 |
| 8-20 | 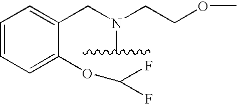 | 548.59 | 549.0 | 5.73 | 2 |
| 8-21 | 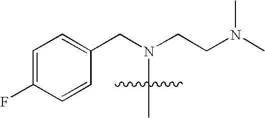 | 513.62 | 514.2 | 4.43 | 2 |
| 8-22 | 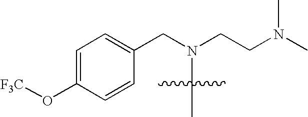 | 579.62 | 580.3 | 5.25 | 2 |
| 8-23 | 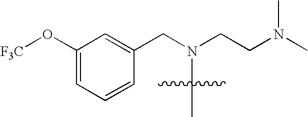 | 579.62 | 580.3 | 5.23 | 2 |
| 8-24 | 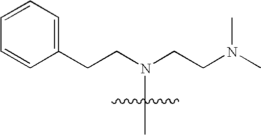 | 509.65 | 510.0 | 4.61 | 2 |
| 8-25 | 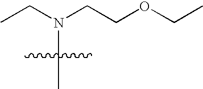 | 434.54 | 435.0 | 4.88 | 2 |
| 8-26 | 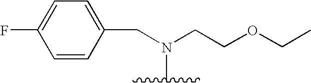 | 514.60 | 515.0 | 5.46 | 2 |
| 8-27 | 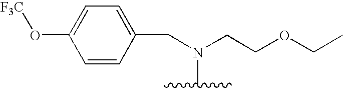 | 580.61 | 581.0 | 5.75 | 2 |
| 8-28 | 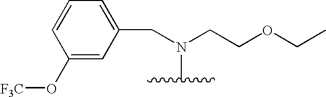 | 580.61 | 581.0 | 5.71 | 2 |
| 8-29 | 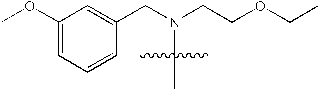 | 526.64 | 527.0 | 5.54 | 2 |
| 8-30 | 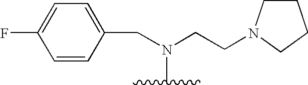 | 539.66 | 540.0 | 7.76 | 2 |
| 8-31 | 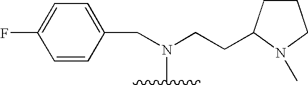 | 553.68 | 554.0 | 4.55 | 2 |
| 8-32 | 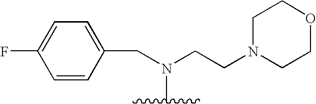 | 555.65 | 556.0 | 4.69 | 2 |
| 8-33 | 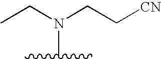 | 415.50 | 416.0 | 4.97 | 2 |
| 8-34 | 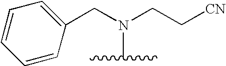 | 477.57 | 478.1 | 5.20 | 2 |
| 8-35 | 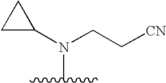 | 427.51 | 428.0 | 4.88 | 2 |
| 8-36 | 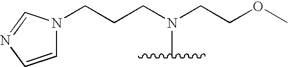 | 500.60 | 501.1 | 3.88 | 2 |
| 8-37 | 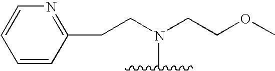 | 497.60 | 498.1 | 4.04 | 2 |
| 8-38 | 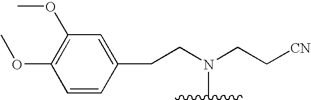 | 551.65 | 552.1 | 5.98 | 2 |
| 8-39 | 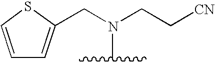 | 483.60 | 484.0 | 6.14 | 2 |
| 8-40 | 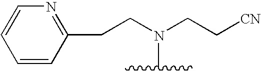 | 492.58 | 493.0 | 4.27 | 2 |
| 8-41 | 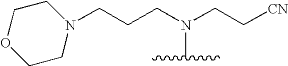 | 514.63 | 515.1 | 3.99 | 2 |
| 8-42 | 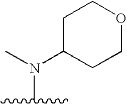 | 432.52 | 433.0 | 4.99 | 2 |
| 8-43 | 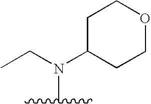 | 446.55 | 447.0 | 5.36 | 2 |

### Example 9

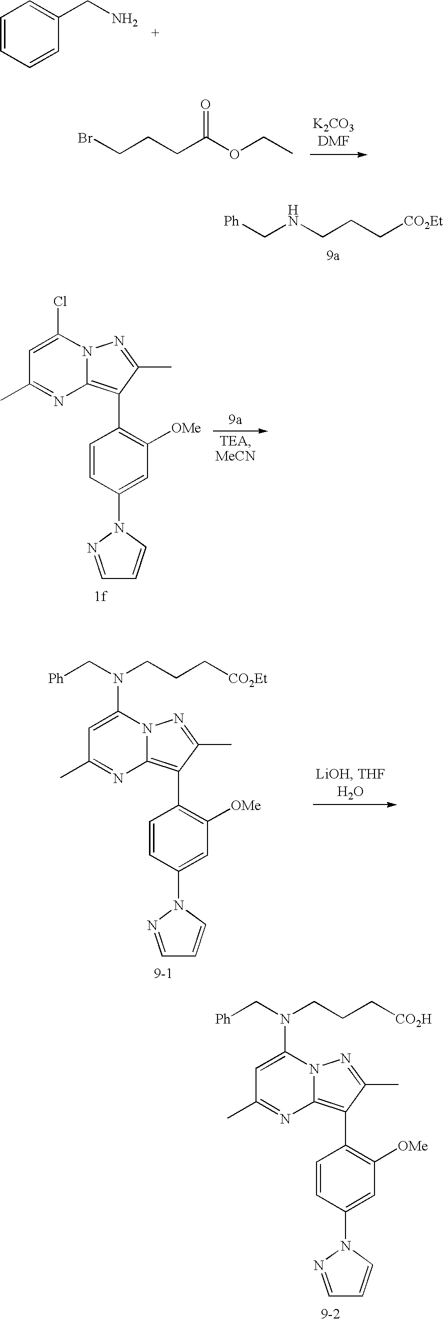 Step 9A:

A mixture of benzylamine (620 mg, 5.8 mmol), ethyl 4-bromobutyrate (750 mg, 3.8 mmol), potassium carbonate (1.6 g, 12 mmol), and DMF (5 mL) was stirred at rt for 17 h. Water was added and the mixture was extracted twice with dichloromethane. The combined organic layers were washed twice with water and once with brine, then were dried over magnesium sulfate, filtered, and evaporated to provide the crude 9a (approximately 1 g) as a gum.

Step, 9B:

A mixture of 1f (50 mg, 0.14 mmol), crude 9A (33 mg, approximately 0.13 mmol), acetonitrile (1 mL), and triethylamine (0.020 mL, 0.16 mmol) was heated in a sealed tube in a microwave reactor at 150° C. for 45 min. The mixture was diluted with methanol and purified directly by preparative HPLC/MS, providing 9-1 (approximately 15 mg) as a TFA salt.

Step 9C:

A solution of 9-1 (10 mg, 0.02 mmol) in 3:1 THF/water (2 mL) was treated with lithium hydroxide hydrate (2.5 mg, 0.06 mmol). The mixture was stirred at rt for 2 h, then was diluted with methanol and purified directly by preparative HPLC/MS, providing 9-2 (4 mg) as a TFA salt.

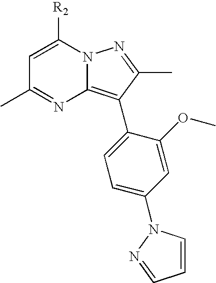

| | | | | t_{R} |
|---|---|---|---|---|
| | R₂ | MW | MS | (method) |
| 9-1 | 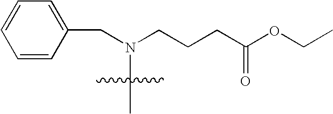 | 538.65 | 539.3 | 6.82 (2) |
| 9-2 | 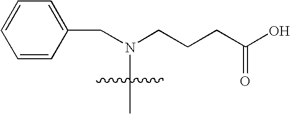 | 510.60 | 511.2 | 5.84 (2) |
| 9-3 | 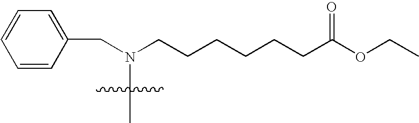 | 580.73 | 581.3 | 7.49 (2) |
| 9-4 | 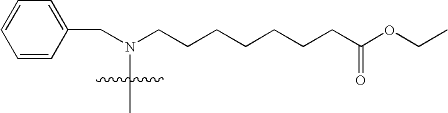 | 594.76 | 595.3 | 7.87 (2) |
| 9-5 | 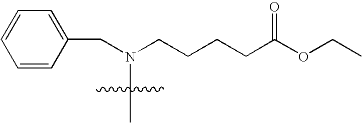 | 552.67 | 553.3 | 6.99 (2) |
| 9-6 | 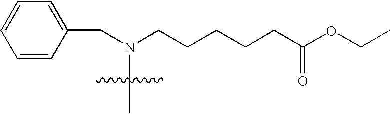 | 566.70 | 567.3 | 5.65 (2) |
| 9-7 | 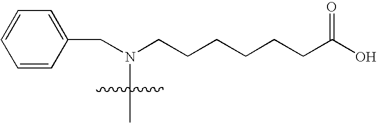 | 552.67 | 553.0 | 6.11 (2) |
| 9-8 | 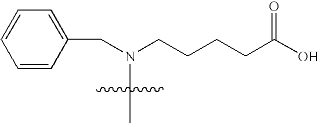 | 524.62 | 525.0 | 5.96 (2) |
| 9-9 | 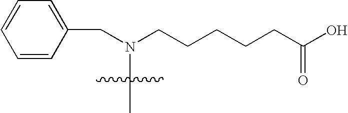 | 538.65 | 539.0 | 6.17 (2) |
| 9-10 | 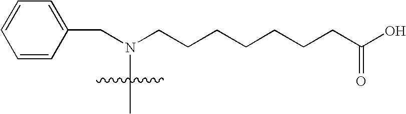 | 566.70 | 567.0 | 5.83 (2) |
| 9-11 | 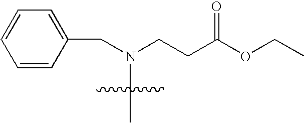 | 524.62 | 525.3 | 6.60 (2) |
| 9-12 | 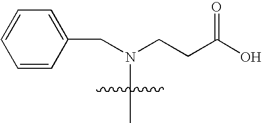 | 496.57 | 497.2 | 5.64 (2) |
| 9-13 | 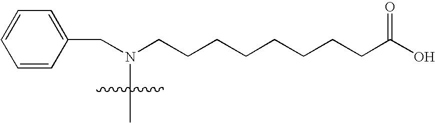 | 580.73 | 581.0 | 5.72 (2) |

### EXAMPLE 10

### Synthesis of Reagent 2-METHYL-4-(PYRAZOL-1-YL)PHENYLBORONIC ACID PINACOL ESTER

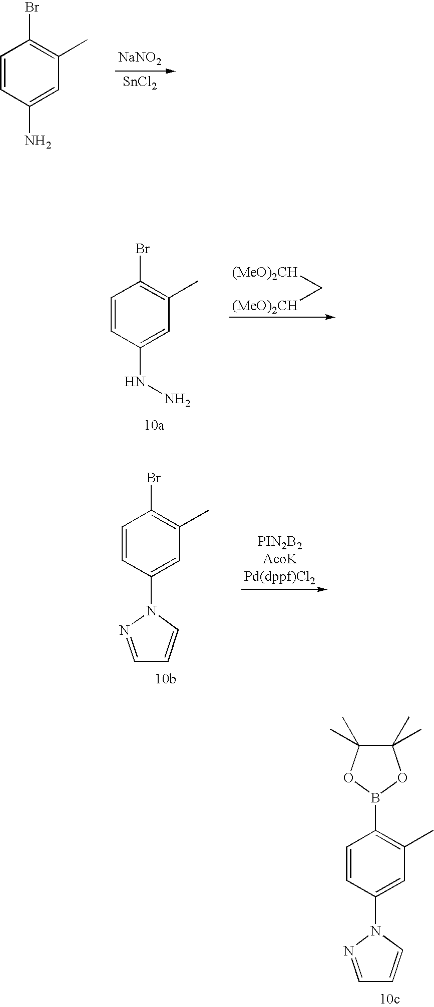 Step 10A:

4-Bromo-3-methylaniline (10.2 g) was suspended in 6N HCl (85 mL) and cooled to 0° C. A solution of sodium nitrite (4 g in 40 mL H₂O) was added over 10 min. The reaction was stirred for 15 min at 0° C. followed by the addition of stannous chloride dihydrate (36 g in 25 mL 12N HCl.) The reaction was stirred for 2 hours at 0° C. The reaction was filtered and the filter cake washed with cold H₂O to afford 4-bromo-3-methylphenylhydrazine hydrochloride 10a (20 g) as a tan solid.

Step 10B:

Compound 10a (20 g) was suspended in 50 mL ethanol. Malondialdehyde bis-dimethylacetal (11.0 mL, 67 mmol) was added and the reaction was heated to 85° C. for 2 hours. The reaction mixture was neutralized with sodium bicarbonate and extracted by washing with DCM. The combined organic layers were dried over magnesium sulfate and concentrated. The residue was taken up in ethyl acetate and the mixture filtered through a pad of Celite®. The filtrate was evaporated, and the oily residue was purified by column chromatography (1:1 ethyl acetate: hexanes) to afford 1-(4-bromo-3-methylphenyl)pyrazole 10b ( 9.6 g, 73%) as an amber oil.

Step 10C:

To a solution of compound 10b (2.0 g in 15 mL dioxane) was added bis(pinacolato)diboron (2.4 g), potassium acetate (2.4 g) and 1,1′-bis(diphenylphosphino) ferrocene dichloropalladium (II) (500 mg). The reaction was heated to 85° C. for 12 hours. The reaction mixture was filtered through a pad of Celite® and the filter cake washed with ethyl acetate. The filtrate was concentrated to a brown liquid which was purified by column chromatography (20% ethyl acetate:hexanes) to afford 2-methyl-4-(pyrazol-1-yl)phenylboronic acid pinacol ester 10c (1.8 g, 75%) as a yellow oil; LC/MS: [M+H]=285.0.

2-Chloro-4-(pyrazol-1-yl)phenylboronic acid pinacol ester 10d was also prepared by the above method.

### Example 11

### Synthesis of Boronic Acid Intermediate

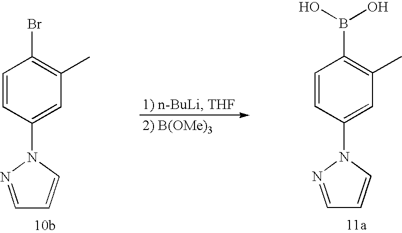 Step 11A:

n-Butyllithium (7.9 mL of a 2.5 M solution in hexanes, 20 mmol) was added to a solution of compound 10b (4.7 g, 20 mmol) in 100 mL THF at −78° C. The mixture was allowed to warm to −25° C. over 1 hr, then the mixture was cooled to −78° C. Trimethylborate (3.4 mL, 30 mmol) was added and the reaction was allowed to warm to RT. Hydrochloric acid (1N, 100 mL) was then added and the mixture was stirred for 16 hr. The pH of the aqueous layer was adjusted to 3-4 using sodium hydroxide and sodium dihydrogen phosphate solution, then the mixture was extracted with ethyl acetate. The organic layer was concentrated and the residue was partitioned between ether and 0.5 N sodium hydroxide solution. The aqueous layer was extracted with two additional portions of ether and was then acidified to pH 3-4 using concentrated hydrochloric acid. The mixture was extracted with ethyl acetate, and the combined ethyl acetate extracts were dried over sodium sulfate, filtered, and evaporated to afford 2-methyl-4-(pyrazol-1-yl)phenylboronic acid (compound 11a, 3.5 g) as an amber gum.

### Example 12

### Synthesis of Boronic Acid Intermediate

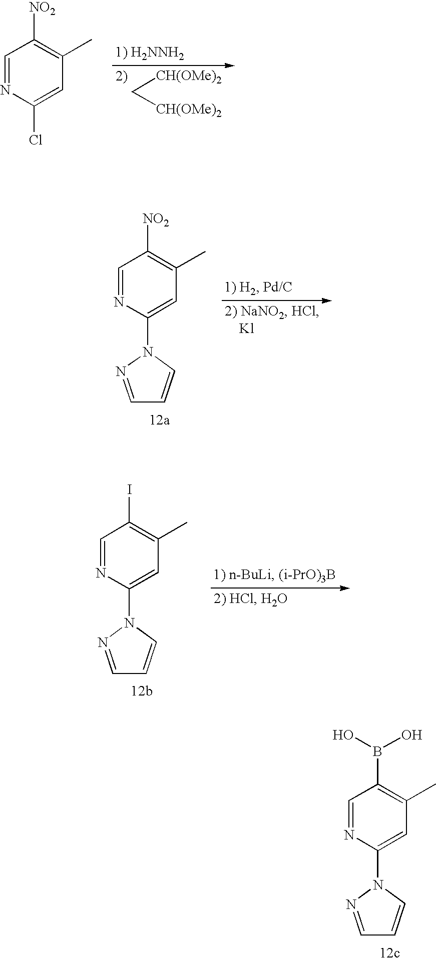 Step 12A:

2-Chloro-4-methyl-5-nitropyridine (5.0 g, 29 mmol, 1.0 eq) was dissolved in 50 mL hydrazine solution (1M solution in THF) and the mixture was stirred and heated in a sealed tube at 80° C. for 22 h. The cooled reaction mixture was filtered, and the solid obtained was washed with ether to provide 5.7 g of a greenish brown solid. A mixture of this solid (5.7 g, 24 mmol, 1.0 eq), malonaldehyde bis(dimethylacetal) (5.9 g, 31 mmol, 1.3 eq), and acetic acid (50 mL) was stirred and heated in a sealed tube at 80° C. for 5 h. The solvent was evaporated, then aqueous sodium bicarbonate solution (200 mL) was added and the mixture was extracted with 2×200 mL ethyl acetate. The combined organic layers were dried over sodium sulfate, filtered, and concentrated. The residue was recrystalized from ethanol to obtain 12a (2.6 g, 53% yield) as a yellow solid.

Step 12B:

A mixture of 12a (2.6 g, 13 mmol) and 10% Pd/C (200 mg) in 30 mL of 1:1 THF/methanol was shaken in a Parr apparatus under 40 psi hydrogen at rt for 2 h. The reaction mixture was filtered through a celite pad and the filtrate concentrated to a light green oil. The oil was resuspended in 10 mL of 3N hydrobromic acid, cooled to 0° C., then treated dropwise with a solution of sodium nitrite (835 mg, 12 mmol, 1.1 eq) in 2 mL water. The mixture was stirred at 0° C. for 1 h, then 2 mL of half-saturated potassium iodide was added and the mixture was stirred at rt for 22 h. Saturated aqueous sodium bicarbonate solution was added, then the mixture was extracted with 2×100 mL ethyl acetate, and the combined organic layers were dried over sodium sulfate, filtered, and concentrated. The residue was purified by silica gel chromatography using 4:1 hexanes/ethyl acetate as eluant, to provide 12b (1.23 g, 33%) as a yellow solid.

Step 12C:

n-Butyllithium (1.8 mL of a 2.0 M solution in pentane, 3.6 mmol) was added dropwise to a solution of compound 12b (600 mg, 2.1 mmol) and triisopropylborate (900 mg, 4.8 mmol) in 5 mL THF at −78° C. The mixture was allowed to warm to rt over 1 hr, then the mixture was cooled to −78° C. and treated with additional triisopropylborate (400 mg, 2.1 mmol), followed by additional n-butyllithium (0.5 mL of a 2.0 M solution in pentane, 1.0 mmol). The mixture again was allowed to warm to rt over 1 h, then 0.8 mL of 1N hydrochloric acid was added and the mixture was stirred for 1 h. The mixture was filtered, rinsing the solid with methanol and ethyl acetate, then the filtrate was concentrated. The residue was chromatographed on silica gel, eluting with 1:1 hexanes/ethyl acetate to provide 12c (220 mg, 52% yield) as a red solid.

### Example 13

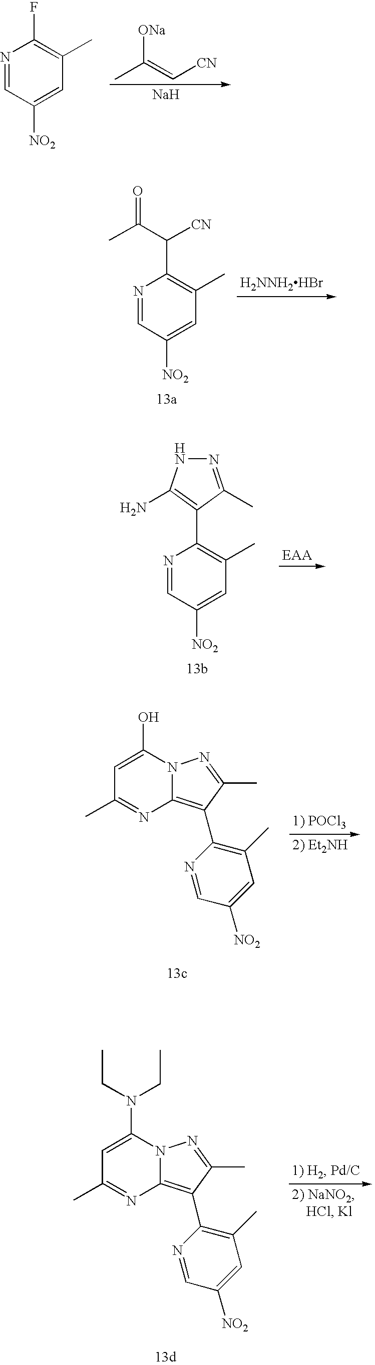 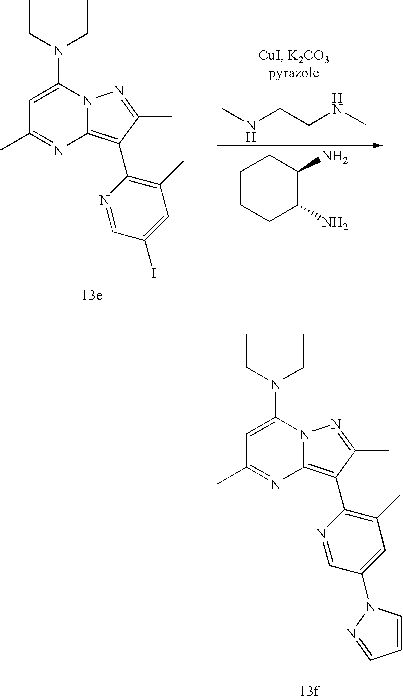 Step 13A:

Sodium hydride (1.54 g of 60% dispersion in oil, 38.5 mmol, 2 eq) was added to a solution of cyanoacetone sodium salt (2.5 g, 23 mmol, 1.2 eq) in DMF (40 mL) at rt. The mixture was stirred for 15 min, then a solution of 2-fluoro-3-methyl-5-nitropyridine (3.0 g, 19.2 mmol, 1.0 eq) in 10 mL DMF was added dropwise. The reaction mixture was stirred at rt for 6 h. The reaction was quenched with 5 g ice, followed by 150 mL water and 10 mL acetic acid. The mixture was extracted with ethyl acetate, then the combined organic extracts were dried over sodium sulfate, filtered, and concentrated. The residue was purified by silica gel chromatography using 30% ethyl acetate in hexanes as eluant, providing 13a (1.85 g, 44% yield) as an orange oil.

Step 13B:

A mixture of 13a (1.8 g, 8.2 mmol, 1.0 eq), hydrazine monohydrobromide (1.0 g, 8.8 mmol, 1.1 eq), ethanol (30 mL) and water (3 mL) was heated at reflux for 17 h. The solvent was evaporated, and the residue was purified directly by silica gel chromatography using 1:1 hexanes/ethyl acetate as eluant, obtaining 13b (1.8 g, 94% yield) as a yellow foam.

Step 13C:

A mixture of 13b (1.8 g, 7.7 mmol, 1.0 eq), ethanol (15 mL), acetic acid (15 mL), and ethyl acetoacetate (1.6 g, 12.4 mmol, 1.6 eq) was heated in a sealed tube at 105° C. for 19 h. The solvent was evaporated, and the residue was deposited on a fritted glass filter, rinsing with ether, to provide 13c (1.0 g, 43% yield) as a yellow solid.

Step 13D:

A mixture of 13c (300 mg, 1.0 mmol, 1.0 eq), phosphorous oxychloride (340 mg, mmol, 2.2 mmol, 2.2 eq), and acetonitrile (10 mL) was refluxed for 3 h. The reaction was poured onto ice, neutralized with aqueous sodium bicarbonate solution, then extracted with ethyl acetate. The combined ethyl acetate extracts were dried over sodium sulfate, filtered and concentrated. Acetonitrile (10 mL) and diethylamine (0.30 mL, 2.9 mmol, 2.9 eq) were added to the residue and the mixture was heated at reflux for 1 h. The mixture was concentrated, then purified directly by silica gel chromatography, eluting with hexanes/ethyl acetate to provide 13d (300 mg, 84% yield).

Step 13E:

10% Pd/C (100 mg) was added to a nitrogen-sparged solution of 13d (200 mg, 0.56 mmol, 1.0 eq) in 6 mL ethanol and 6 mL THF. The mixture was shaken in a Parr shaker under 35 psi hydrogen gas at rt for 2 h. The mixture was purged with nitrogen and filtered. The filtrate was concentrated to provide the crude aminopyridine. To an ice-cold solution of this crude aminopyridine (entire amount) in 4N hydrochloric acid (5 mL) was added dropwise a solution of sodium nitrite (43 mg, 0.62 mmol, 1.1 eq) in water (1 mL). The mixture was stirred at 0° C. for 1 h, followed by dropwise addition of a solution of potassium iodide (150 mg, 0.90 mmol, 1.6 eq) in 1.5 mL water. The mixture was stirred at rt for 2 h, then 20 mL saturated aqueous sodium bicarbonate solution was added and the mixture was extracted with ethyl acetate. The combined organic layers were dried over sodium sulfate, filtered, and concentrated. The residue was purified by silica gel chromatography using 95:5:0.01 chloroform/methanol/aqueous ammonia as eluant, providing 13e (73 mg, 27% yield).

Step 13F:

To a solution of 13e (20 mg, 0.05 mmol, 1.0 eq) in dioxane (1 mL) were added potassium carbonate (14 mg, 0.1 mmol, 2.0 eq), pyrazole (6 mg, 0.09 mmol, 1.8 eq), copper(I) iodide (6 mg, 0.03 mmol, 0.6 eq), trans-1,2-diaminocyclohexane (5 mg, 0.04 mmol, 0.8 eq), and N,N′-dimethylethylenediamine (5 mg, 0.06 mmol, 1.1 eq). The mixture was stirred and heated in a sealed tube at 90° C. for 16 h. The reaction mixture was filtered through a celite pad, concentrated, and purified by prep HPLC/MS to obtain 13-1 (7 mg, 30% yield) as a TFA salt.

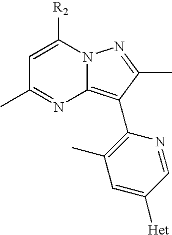

| | | | | | t_{R} |
|---|---|---|---|---|---|
| | R₂ | Het | MW | MS | (method) |
| 13-1 | 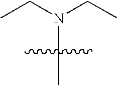 | 1-pyrazolyl | 375.48 | 376 | 4.47 (2) |
| 13-2 | 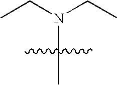 | 3-trifluoromethyl- 1-pyrazolyl | 443.48 | 444 | 5.00 (2) |
| 13-3 | 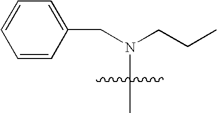 | 1-pyrazolyl | 451.57 | 452 | 5.37 (2) |

### EXAMPLE 14

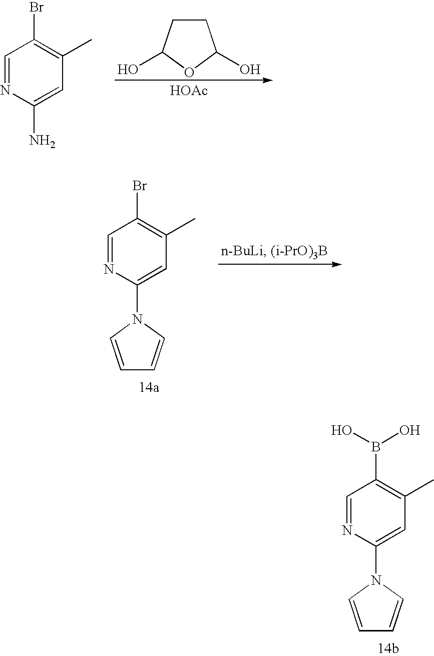 Step 14A:

A solution of 2-amino-5-bromo-4-methylpyridine (1 g, 5.4 mmol) and 2,5-dihydroxytetrahydrofuran (2.8 g, 27 mmol) in acetic acid (10 mL) was heated at 90° C. in a sealed tube for 2 h. The reaction mixture was concentrated and the residue was purified by silica gel chromatography using 4:1 hexanes/ethyl acetate, providing 14a (900 mg, 71% yield) as a light yellow oil.

Step 14B:

n-Butyllithium (3.6 mL of a 2.0 M solution in pentane, 7.2 mmol) was added dropwise to a solution of compound 14a (860 mg, 3.6 mmol) and triisopropylborate (1.4 g, 7.3 mmol) in 6 mL THF at −78° C. The mixture was allowed to warm to rt over 1 h, then 0.5 mL of 4N hydrochloric acid was added and the mixture was stirred for 10 min. The mixture was extracted with 2×25 mL dichloromethane, then the organic layer was dried over sodium sulfate, filtered, and concentrated to provide 14b (250 mg) as a yellow oil. The aqueous layer was concentrated, then the solid residue was washed with ethanol. The combined ethanol filtrates were concentrated to provide additional 14b (500 mg) as a yellow oil.

### Example 15

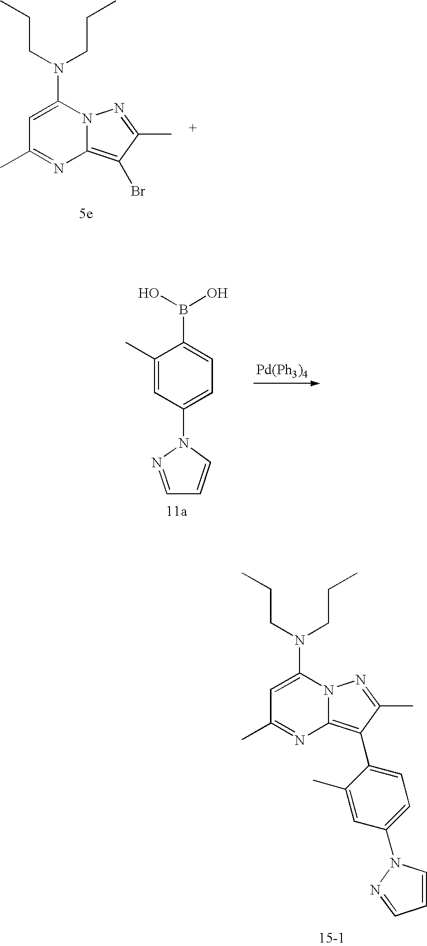 Step 15A:

Tetrakis(triphenylphosphine)palladium(0) (46 mg, 0.04 mmol) was added to a solution of 5e (165 mg, 0.51 mmol) and 11a (80 mg, 0.40 mmol) in 2:1 toluene/ethanol (2 mL). Aqueous 2.0 M sodium carbonate solution (0.6 mL, 1.2 mmol) was added and the mixture was stirred and heated at 90° C. for 3h in a sealed vial. The cooled mixture was extracted with ethyl acetate, then the combined organic extracts were washed with brine, dried over sodium sulfate, filtered, and concentrated. The residue was purified by silica gel chromatography, eluting with 2:1 hexanes/ethyl acetate. Two thirds of the resulting partially-purified product was again chromatographed on silica gel, providing 15-1 (6 mg) as an oil.

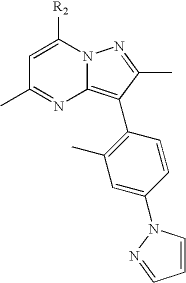

| | | | | t_{R} |
|---|---|---|---|---|
| | R₂ | MW | MS | (method) |
| 15-1 | 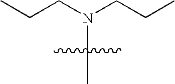 | 402.54 | 403.4 | 2.01 (1) |
| 15-2 | 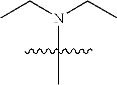 | 374.49 | 375.1 | 1.84 (1) |

### Example 16

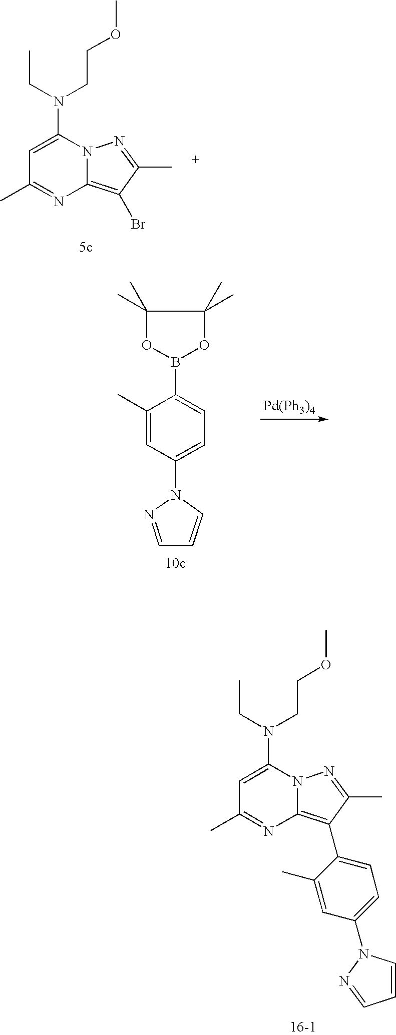 Step 16A:

Tetrakis(triphenylphosphine)palladium(0) (30 mg, 0.026 mmol) was added to a solution of 5c (164 mg, 0.50 mmol) and 10c (284 mg, 1.0 mmol) in 10:1 dioxane/water (20 mL). Potassium carbonate (207 mg, 1.5 mmol) was added and the mixture was stirred and heated at 100° C. for 16 h in a sealed vial. The cooled mixture was extracted with ethyl acetate, then the combined organic extracts were dried over sodium sulfate, filtered, and concentrated. The residue was purified by preparative HPLC/MS to provide 16-1 (81 mg, 31% yield) as a TFA salt.

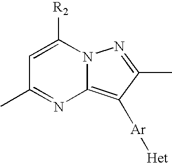

| | | | | | t_{R} |
|---|---|---|---|---|---|
| | R₂ | —Ar—Het | MW | MS | (method) |
| 16-1 | 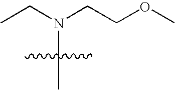 | 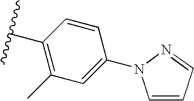 | 404.51 | 405 | 5.02 (2) |
| 16-2 | 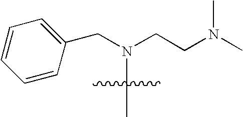 | 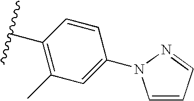 | 479.63 | 479 | 4.56 (2) |
| 16-3 | 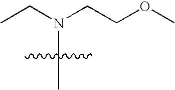 | 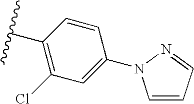 | 424.93 | 424 | 5.19 (2) |
| 16-4 | 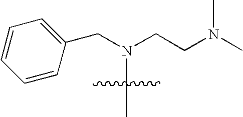 | 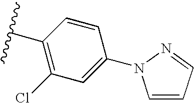 | 500.05 | 499 | 4.70 (2) |
| 16-5 | 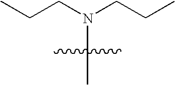 | 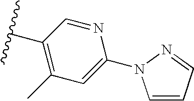 | 403.53 | 404 | 6.31 (2) |
| 16-6 | 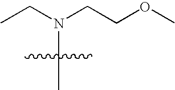 | 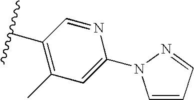 | 405.50 | 406 | 5.19 (2) |
| 16-7 | 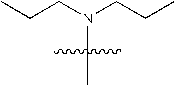 | 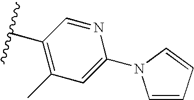 | 402.54 | 403 | 6.84 (2) |

### Example 17

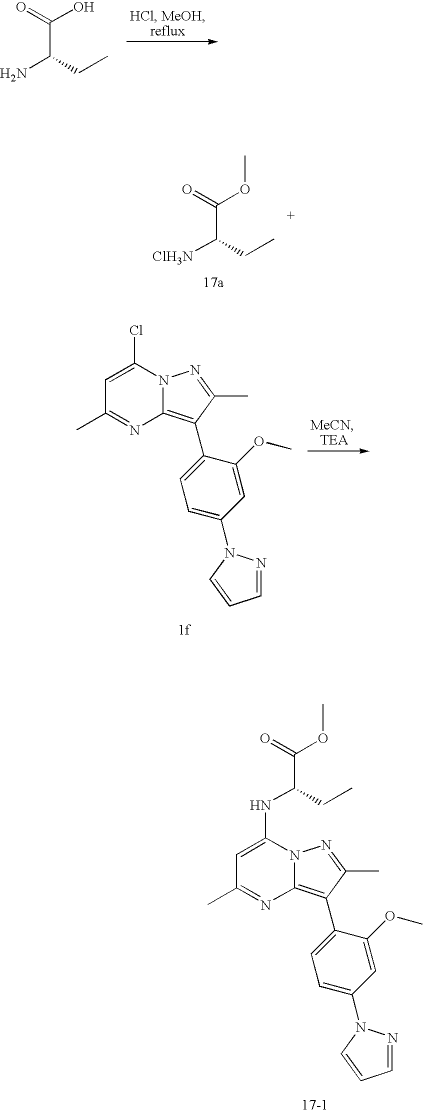 Step 17A:

Acetyl chloride (20 mL, 280 mmol) was added to methanol (200 mL) with stirring in an ice bath. (S)-2-Aminobutyric acid (10.0 g, 97 mmol) was added to the methanol solution, and the mixture was heated to reflux for 64 h. The cooled solution was evaporated to dryness, then the residue was co-evaporated three times with toluene, then dried under vacuum to provide 17a (14.8 g) as a white solid.

Step 17B:

A mixture of 17a (98 mg, 0.65 mmol), 1F (150 mg, 0.42 mmol), triethylamine (0.088 ml, 0.63 mmol), and acetonitrile (1.5 ml) was heated at 150° C. in a microwave reactor for 35 min. The mixture was partitioned between ethyl acetate and aqueous sodium bicarbonate, then the organic layer was dried over sodium sulfate, filtered, and concentrated. The residue was chromatographed on silica gel, eluting with 1:1 hexanes/ethyl acetate to provide 17-1 (70 mg) as a tan solid. HPLC-MS (method 2) t_{R}=5.07 MH₊=435.0

### EXAMPLE 18

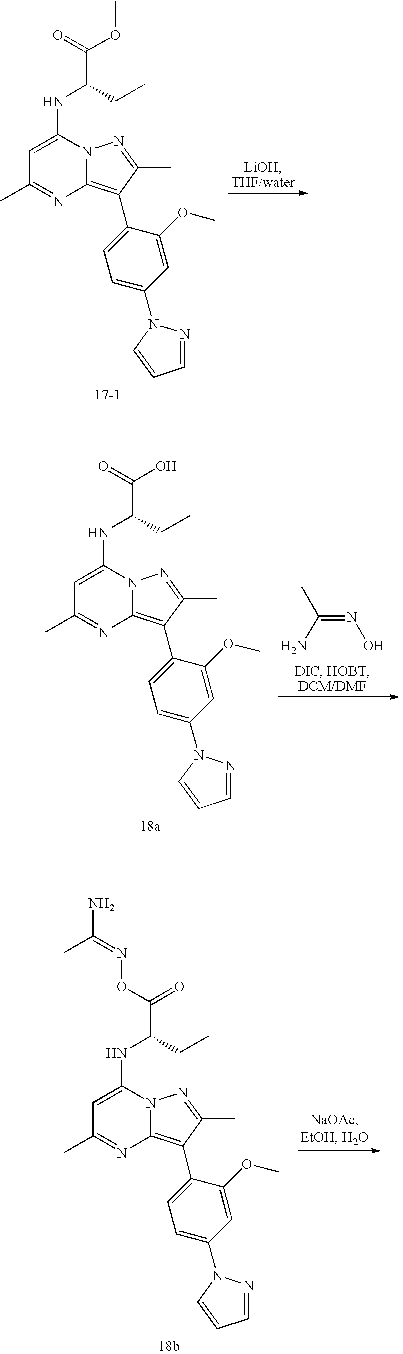 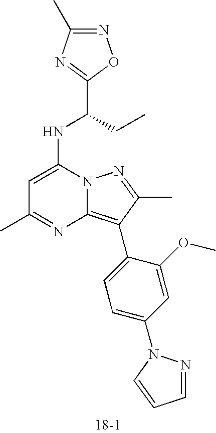 Step 18A:

Lithium hydroxide hydrate (10 mg, 0.23 mmol) was added to a mixture of 17-1 (65 mg, 0.15 mmol) THF (2 mL), and water (1 mL). The mixture was stirred vigorously at rt for 90 min, then the mixture was acidified with 2N hydrochloric acid (0.12 mL, 0.24 mmol). The solvent was evaporated. The solid residue was washed with water, co-evaporated with toluene, then dried under vacuum to provide 18a as a gummy solid.

Step 18B:

A mixture of 18a (entire amount), HOBT (27 mg, 0.20 mmol), acetamide oxime (15 mg, 0.21 mmol), and dichloromethane (2 mL) was treated with DIC (0.030 mL, 0.20 mmol) at rt. DMF (0.25 mL) was added and the mixture was stirred for 10 min, then was concentrated to dryness. Ethyl acetate was added and the mixture was washed successively with saturated aqueous sodium bicarbonate, water, and brine. The ethyl acetate layer was dried over sodium sulfate, filtered, and concentrated to provide crude 18b.

Step 18C:

Sodium acetate (28 mg, 0.30 mmol) was added to a solution of crude 18b (entire amount) in 5:1 ethanol/water (1.2 mL), and the mixture was heated in a sealed tube at 75° C. for 1.5 h. The solvent was evaporated. The residue was partitioned between dichloromethane and aqueous sodium bicarbonate, then the organic layer was dried over sodium sulfate, filtered, and concentrated. The residue was chromatographed on silica gel, eluting was 2:3 hexanes/ethyl acetate to provide 18-1 (30 mg, 44% yield from 17-1). HPLC-MS (methold 2) t_{R}=4.97 MH⁺=459.0

### Example 19

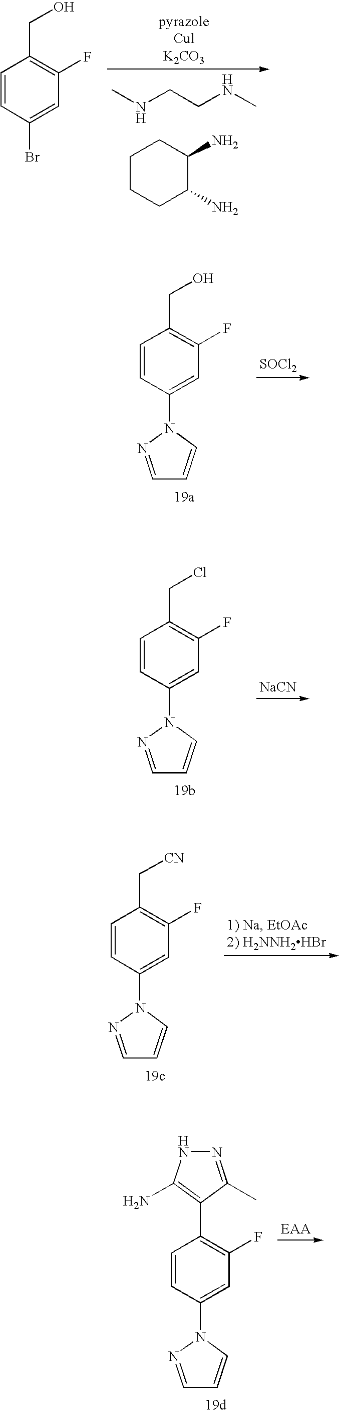 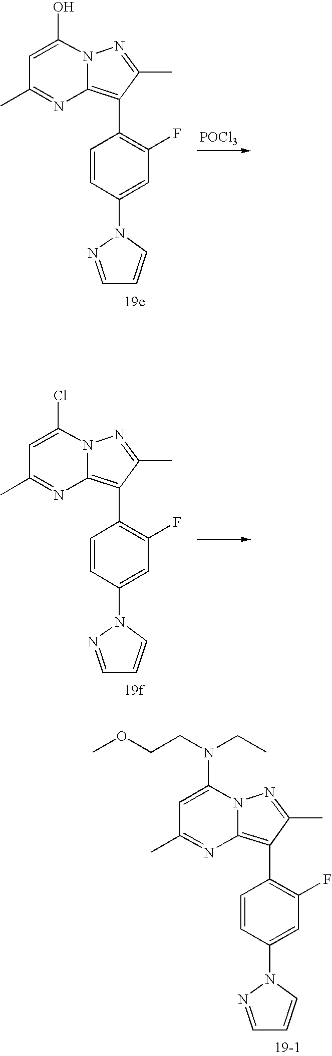 Step 19A:

A mixture of 4-bromo-2-fluorobenzyl alcohol (9.71 g, 47 mmol), CuI (8.9 g, 47 mmol), N,N′-dimethylethylenediamine (0.44 mL), trans-1,2-diaminocyclohexane (0.52 mL), pyrazole (4.7 g, 69 mmol), and potassium carbonate (64 g, 460 mmol) in dioxane (200 mL) was heated at 100° C. for 19 h. The cooled mixture was filtered, then the filtrate was evaporated. The residue was taken up in ethyl acetate and the organic mixture was washed with water and brine, then dried over sodium sulfate and filtered. Concentration provided 19a, the entire amount of which was used in the next reaction step.

Step 19B:

Thionyl chloride (6.9 mL, 95 mmol) was added dropwise to a solution of 19a (entire amount from previous step) in dichloromethane (50 mL) and the mixture was refluxed for 2.5 h. The cooled mixture was poured onto ice-water and extracted with dichloromethane. The combined organic layers were dried over sodium sulfate, filtered, and concentrated to provide 19b (12 g) as a brown solid.

Step 19C:

DMSO (10 mL) was added to a mixture of crude 19b (12 g) and sodium cyanide (2.3 g, 47 mmol) and the resulting suspension was stirred and heated at 80° C. for 45 min. DMSO was removed under vacuum, then the residue was chromatographed on silica gel, eluting with hexanes/ethyl acetate to provide 19c (2.2 g).

Step 19D:

To a solution of 19c (2.2 g, 10 mmol) in ethyl acetate (50 mL) was added metallic sodium (400 mg, 17 mmol) portionwise, and the mixture was heated at 70° C. for 16 h. The resulting suspension was decanted onto ice-water and the mixture was acidified to pH 4 with hydrochloric acid. The organic phase was dried over sodium sulfate, filtered, and concentrated. The residue was taken up in 6:1 ethanol/water (50 mL), then hydrazine monohydrobromide (4.52 g, 41 mmol) was added and the mixture was stirred and refluxed for 22 h. The mixture was concentrated, then taken up in ethyl acetate and washed with water and brine. The organic phase was dried over sodium sulfate, filtered, and evaporated to dryness to yield crude 19d, the entire amount of which was used in the next reaction step.

Step 19E:

A suspension of 19d (entire amount from previous step) and ethyl acetoacetate (2.1 g, 16 mmol) in 1:1 ethanol/acetic acid (10 mL) was refluxed for 18 h. The solvents were evaporated, then the residue was deposited onto a fritted glass filter and washed with ether to provide 19e (600 mg) as a solid.

Step 19F:

To a suspension of 19e (600 mg, 1.85 mmol) in dioxane (2.5 mL) was added triethylamine (0.52 mL, 3.7 mmol) and phosphorus oxychloride (0.43 mL, 4.6 mmol), and the mixture was refluxed for 1 h. The cooled mixture was poured onto ice-water, then was extracted with ethyl acetate. The combined organic extracts were dried over sodium sulfate, filtered, and concentrated to provide 19f (500 mg), which was used without further purification.

Step 19G:

A suspension of 19f (57 mg, 0.17 mmol) and (2-methoxyethyl)ethylamine (0.031 mL, 0.25 mmol) in acetonitrile (0.5 mL) was heated in a sealed tube at 160° C. in a microwave reactor for 16 min. The crude mixture was subjected to purification by preparative HPLC/MS to provide 19-1 (17 mg) as a TFA salt.

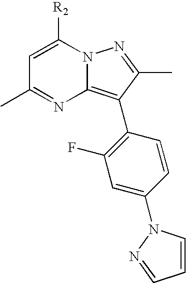

| | | | | t_{R} |
|---|---|---|---|---|
| | R₂ | MW | MS | (method) |
| 19-1 | 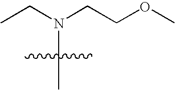 | 408.48 | 409.0 | 5.35 (2) |
| 19-2 | 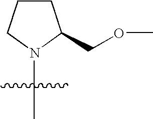 | 420.49 | 421.0 | 5.34 (2) |

### Example 20

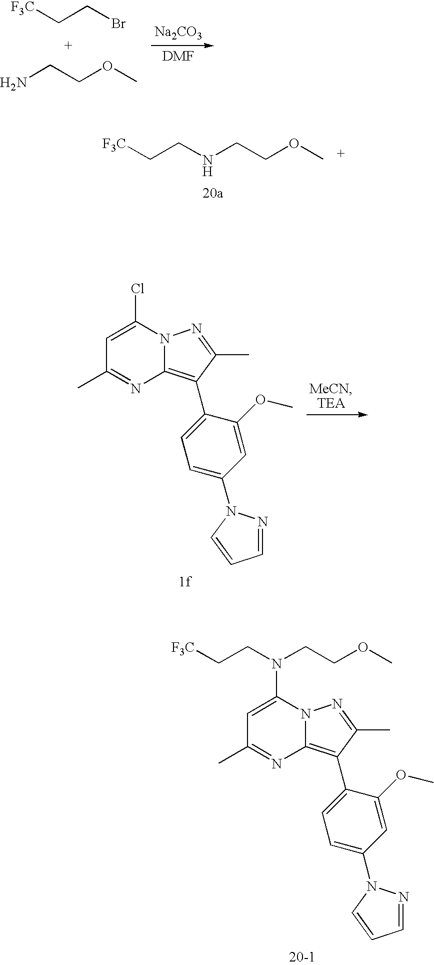 Step 20A:

Sodium carbonate (500 mg, 4.7 mmol) was added to a solution of 2-methoxyethylamine (0.20 mL, 2.3 mmol) and 3-bromo-1,1,1-trifluoropropane (0.40 mL, 3.8 mmol) in DMF (2 mL). The mixture was stirred at rt for 48 h, then water was added and the mixture was extracted with dichloromethane. The combined organic layers were washed with brine, dried over sodium sulfate, filtered, and concentrated under vacuum to provide 20a as an oil.

Step 20B:

Reaction of one half of the crude 20a with 1f (30 mg) according to the procedure of the final step of Example 1 provided 20-1 (13 mg) as a TFA salt following preparative HPLC/MS purification.

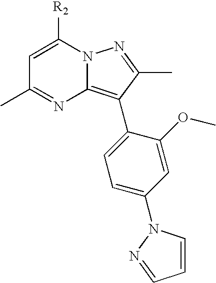

| | | | | t_{R} |
|---|---|---|---|---|
| | R₂ | MW | MS | (method) |
| 20-1 | 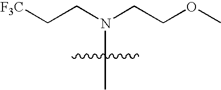 | 488.51 | 489.1 | 1.88 (4) |
| 20-2 | 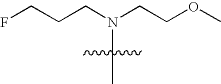 | 452.53 | 453.2 | 1.81 (4) |

### Example 21

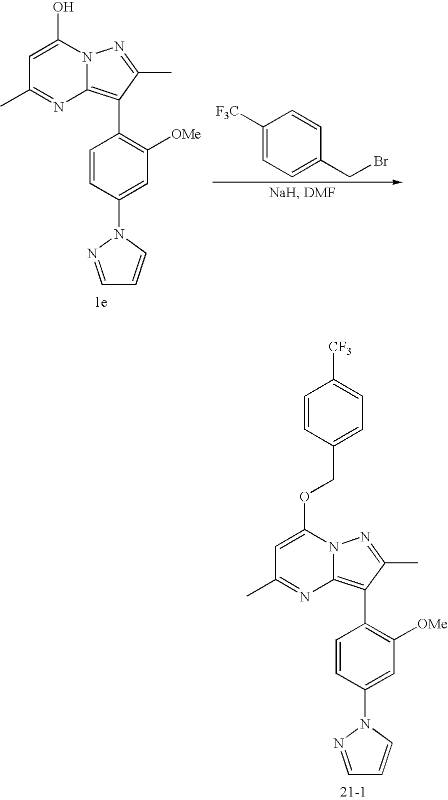 Step 21A:

To solution of 1e (30 mg, 0.09 mmol) in DMF (2 mL) was added sodium hydride (approximately 10 mg of 60% dispersion in mineral oil, 0.25 mmol), and the mixture was stirred at rt for 5 min. 4-Fluorobenzyl bromide (approximately 100 mg, 0.30 mmol) was added and the mixture was stirred in a sealed vial at rt for 2 h. The mixture was purified directly using preparative HPLC/MS to provide compound 21-1 (8 mg) as a TFA salt. HPLC-MS (method 2) t_{R}=1.49 MH⁺=444.1

### Example 22

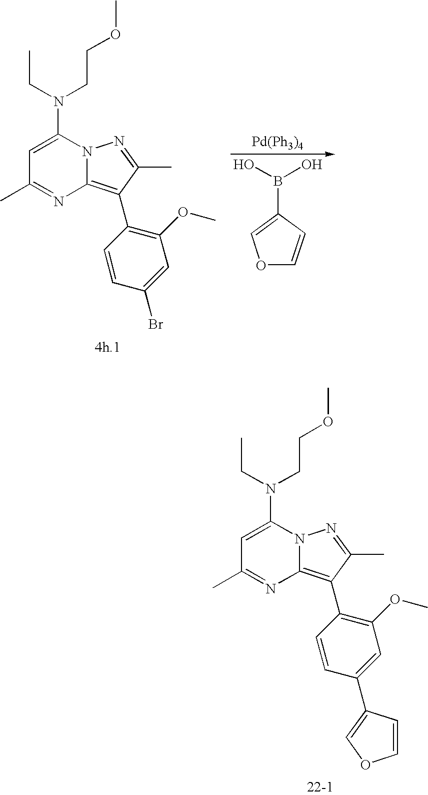 Step 22A:

Tetrakis(triphenylphosphine)palladium(0) (15 mg, 0.013 mmol) was added to a solution of 4h.1 (50 mg, 0.12 mmol) and furan-3-boronic acid (23 mg, 0.21 mmol) in dioxane (1 mL). A solution of potassium carbonate (40 mg, 0.29 mmol) in water (0.20 mL) was added and the mixture was stirred and heated at 100° C. for 16 h in a sealed vial. The cooled mixture was diluted with methanol, filtered, and purified directly by preparative HPLC/MS to provide 22-1 (22 mg, 34% yield) as a TFA salt.

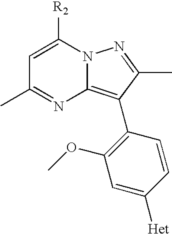

| | | | | | t_{R} |
|---|---|---|---|---|---|
| | R₂ | Het | MW | MS | (method) |
| 22-1 | 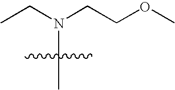 | 3-furanyl | 420.51 | 420.8 | 1.49 (4) |
| 22-2 | 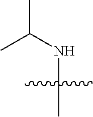 | 3,5-dimethyl-4-oxazolyl | 405.50 | 406 | 1.48 (4) |
| 22-3 | 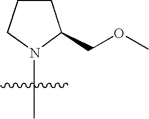 | 4-pyrazolyl | 432.52 | 433 | 6.21 (2) |
| 22-4 | 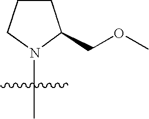 | 3-pyridyl | 443.55 | 444 | 4.05 (2) |
| 22-5 | 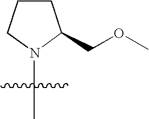 | 1-methyl-4-pyrazolyl | 446.55 | 447 | 6.30 (2) |
| 22-6 | 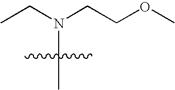 | 3-thienyl | 436.58 | 436.8 | 1.60 (4) |
| 22-7 | 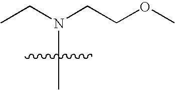 | 2-furanyl | 420.51 | 420.9 | 1.60 (4) |
| 22-8 | 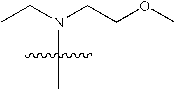 | 2-thienyl | 436.58 | 437 | 6.50 (2) |
| 22-9 | 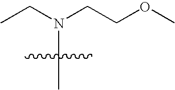 | 1-(tert-butyloxycarbonyl)- 2-pyrrolyl | 519.64 | 520.1 | 7.27 (2) |

### Example 23

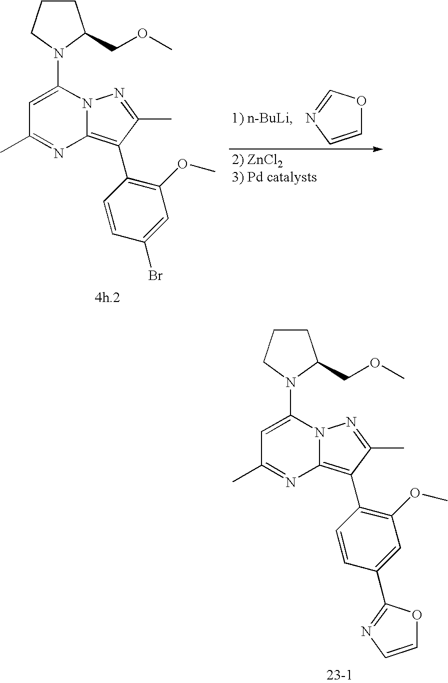 Step 23A:

n-Butyllithium (0.80 mL of 2.5 M solution in hexanes, 2.0 mmol) was added dropwise to a solution of oxazole (0.138 mL, 2.0 mmol) in THF (10 mL) at −78° C. After 45 min, zinc chloride (8 mL of a 0.5 M solution in THF, 4.0 mmol) was added and the mixture was warmed to 0° C. and stirred at that temp for 1 h. A solution of 4h.2 (91 mg, 0.20 mmol) in THF (2.5 mL) was added, followed by tetrakis(triphenylphosphine)palladium(0) (46 mg, 0.04 mmol). The mixture was refluxed for 1.5 h, then dichlorobis(triphenylphosphine)palladium(II) (25 mg, 0.036 mmol) was added and the mixture was refluxed for an additional 1.5 h. Aqueous sodium bicarbonate solution was added, and the mixture was extracted with ethyl acetate. The combined ethyl acetate extracts were dried over sodium sulfate, filtered, and concentrated, then the residue was partially purified by silica gel chromatography using hexanes/ethyl acetate as eluant. The partially purified product was applied as a methanol solution to a cation exchange column (500 mg Varian SCX, H⁺ form, pre-washed with dichloromethane and methanol). Elution of impurities with methanol, followed by elution of the product with 1M ammonia in methanol, gave 23-1 (18 mg, 21% yield) as a tan solid. HPLC-MS (method 2) t_{R}=4.92 MH⁺=434.0

### Example 24

### CRF Receptor Binding Activity

The compounds of this invention may be evaluated for binding activity to the CRF receptor by a standard radioligand binding assay as generally described by Grigoriadis et al. (Mol. Pharmacol vol 50, pp 679-686, 1996) and Hoare et al. (Mol. Pharmacol vol 63 pp 751-765, 2003.) By utilizing radiolabeled CRF ligands, the assay may be used to evaluate the binding activity of the compounds of the present invention with any CRF receptor subtype.

Briefly, the binding assay involves the displacement of a radiolabeled CRF ligand from the CRF receptor. More specifically, the binding assay is performed in 96-well assay plates using 1-10 μg cell membranes from cells stably transfected with human CRF receptors. Each well receives about 0.05 ml assay buffer (e.g., Dulbecco's phosphate buffered saline, 10 mM magnesium chloride, 2 mM EGTA) containing compound of interest or a reference ligand (for example, sauvagine, urocortin I or CRF), 0.05 ml of [¹²⁵I] tyrosine-sauvagine (final concentration ˜150 pM or approximately the K_{D} as determined by Scatchard analysis) and 0.1 ml of a cell membrane suspension containing the CRF receptor. The mixture is incubated for 2 hours at 22° C. followed by separation of the bound and free radioligand by rapid filtration over glass fiber filters. Following three washes, the filters are dried and radioactivity (Auger electrons from ¹²⁵I) is counted using a scintillation counter. All radioligand binding data may be analyzed using the non-linear least-squares curve-fitting programs Prism (GraphPad Software Inc) or XLfit (ID Business Solutions Ltd).

### Example 25

### CRF-Stimulated Adenylate Cyclase Activity

The compounds of the present invention may also be evaluated by various functional testing. For example, the compounds of the present invention may be screened for CRF-stimulated adenylate cyclase activity. An assay for the determination of CRF-stimulated adenylate cyclase activity may be performed as generally described by Battaglia et al. (Synapse 1:572, 1987) with modifications to adapt the assay to whole cell preparations.

More specifically, the standard assay mixture may contain the following in a final volume of 0.1 ml: 2 mM L-glutamine, 20 mM HEPES, and 1 mM IMBX in DMEM buffer. In stimulation studies, whole cells with the transfected CRF receptors are plated in 96-well plates and incubated for 30 min at 37° C. with various concentrations of CRF-related and unrelated peptides in order to establish the pharmacological rank-order profile of the particular receptor subtype. Following the incubation, cAMP in the samples is measured using standard commercially available kits, such as cAMP-Screen™ from Applied Biosystems. For the functional assessment of the compounds, cells and a single concentration of CRF or related peptides causing 50% stimulation of cAMP production are incubated along with various concentrations of competing compounds for 30 min at 37° C., and cAMP determined as described above.

It will be appreciated that, although specific embodiments of the invention have been described herein for purposes of illustration, various modifications may be made without departing from the spirit and scope of the invention. Accordingly, the invention is not limited except as by the appended claims.

What is claimed is:

1. A compound having the following structure: 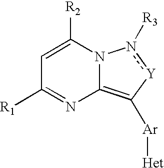
or a pharmaceutically acceptable salt, ester, solvate, stereoisomer, or prodrug thereof,
wherein:
“---” represents the second bond of an optional double bond;
R₁ is hydrogen, alkyl, substituted alkyl, —NH₂, or halogen;
R₂ is —NR₇R₈ or —OR₁₀;
R₃ is null, hydrogen, or alkyl;
Y is ═(CR₄)— or —(C═O)—;
R₄ is hydrogen, alkyl, substituted alkyl, thioalkyl, alkylsulfinyl, or alkylsulfonyl;
Ar is phenyl, phenyl optionally substituted with 1 or 2 R₅, pyridyl, or pyridyl optionally substituted with 1 or 2 R₅;
R₅ at each occurrence is alkyl, substituted alkyl, alkoxy, substituted alkoxy, cyano, halogen, alkylsulfinyl, or alkylsulfonyl;
Het is heteroaryl optionally substituted with 1 or 2 R₆;
R₆ at each occurrence is alkyl, substituted alkyl, alkoxy, substituted alkoxy, cyano, halogen, —C(O)OR₁₁, or hydroxy;
R₇ is hydrogen, alkyl, substituted alkyl, heterocycle, substituted heterocycle, heterocyclealkyl, substituted heterocyclealkyl, alkoxyalkyl, substituted alkoxyalkyl, aryl, substituted aryl, aryloxyalkyl, substituted aryloxyalkyl, arylalkyl, or substituted arylalkyl;
R₈ is alkyl, substituted alkyl, heterocycle, substituted heterocycle, heterocyclealkyl, substituted heterocyclealkyl, alkoxyalkyl, substituted alkoxyalkyl, aryl, substituted aryl, aryloxyalkyl, substituted aryloxyalkyl, arylalkyl, or substituted arylalkyl; or
R₇ and R₈, together with the nitrogen atom to which they are attached, form a heterocycle which is optionally substituted by 1, 2, or 3 R₉;
R₉ at each occurrence is hydroxy, alkylsulfonyl, alkylsulfinyl, —CH₂—OC(O)R₁₃, —C(O)OR₁₁, —C(O)NR₁₁R₁₂, alkyl, substituted alkyl, alkoxy, substituted alkoxy, arylalkyl, substituted arylalkyl, heteroarylalkyl, substituted heteroarylalkyl, aryl, substituted aryl, heterocycle, substituted heterocycle, alkoxyalkyl, or substituted alkoxyalkyl;
R₁₀ is alkyl, substituted alkyl, arylalkyl, substituted arylalkyl, heteroarylalkyl, substituted heteroarylalkyl, aryloxyalkyl, or substituted aryloxyalkyl;
R₁₁, and R₁₂ are the same or different and independently hydrogen, alkyl, substituted alkyl, heterocycle, substituted heterocycle, heterocyclealkyl, substituted heterocyclealkyl, alkoxyalkyl, substituted alkoxyalkyl, aryl, substituted aryl, aryloxyalkyl, substituted aryloxyalkyl, arylalkyl, or substituted arylalkyl; and
R₁₃ is alkyl, substituted alkyl, heterocycle, substituted heterocycle, alkoxy, substituted alkoxy.

2. The compound of claim 1 wherein R₁ is hydrogen, alkyl, or substituted alkyl.

3. The compound of claim 1 wherein R₂ is —NR₇R₈.

4. The compound of claim 3 wherein R₇ and R₈ together with the nitrogen atom to which they are attached form a heterocycle substituted by 1 R₉.

5. The compound of claim 4 where R₉ is hydroxy, alkylsulfonyl, alkylsulfinyl, alkyl, substituted alkyl, arylalkyl, substituted arylalkyl, heteroarylalkyl, substituted heteroarylalkyl, aryl, substituted aryl, heterocycle, substituted heterocycle, alkoxyalkyl, or substituted alkoxyalkyl.

6. The compound of claim 1 wherein R₃ is null.

7. The compound of claim 6 wherein Y is ═(CR₄)—.

8. The compound of claim 7 wherein R₄ is hydrogen, alkyl, or substituted alkyl.

9. The compound of claim 1 wherein R₃ is hydrogen or alkyl.

10. The compound of claim 9 wherein Y is —(C═O)—.

11. The compound of claim 1 wherein Ar is substituted by 1 R₅.

12. The compound of claim 11 wherein R₅ is alkyl, substituted alkyl, alkoxy, substituted alkoxy, cyano, or halogen.

13. The compound of claim 1 wherein Het is substituted by 1 R₆.

14. The compound of claim 13 wherein R₆ is alkyl, substituted alkyl, alkoxy, substituted alkoxy, cyano, or halogen.

15. The compound of claim 1 wherein R₂ is —OR₁₀.

16. The compound of claim 3 wherein Y is ═(CR₄)—.

17. The compound of claim 16 wherein R₅ is alkyl, substituted alkyl, alkoxy, or substituted alkoxy.

18. The compound of claim 17 wherein R₈ is alkyl, substituted alkyl, heteroarylalkyl, substituted heteroarylalkyl, alkoxyalkyl, substituted alkoxyalkyl, aryloxyalkyl, substituted aryloxyalkyl, arylalkyl, or substituted arylalkyl.

19. The compound of claim 18 wherein R₇ is hydrogen, alkyl, substituted alkyl, or alkoxyalkyl.

20. A composition comprising a compound of claim 1 and a pharmaceutically acceptable carrier or diluent.

21. A method for treating a disorder manifesting hypersecretion of CRF in a mammal comprising administering to the animal an effective amount of the pharmaceutical composition of claim 20.

22. The method of claim 21 wherein the disorder is stroke.

23. The method of claim 21 wherein the disorder is depression.

24. The method of claim 21 wherein the disorder is obsessive-compulsive disorder.

25. The method of claim 21 wherein the disorder is irritable bowel syndrome.
